(19) United States
(12) Patent Application Publication
(10) Pub. No.: US 2025/0244718 A1
(43) Pub. Date: Jul. 31, 2025

(54) ELECTRONIC DEVICE

(71) Applicant: Apple Inc., Cupertino, CA (US)

(72) Inventors: Richard A. Davis, San Carlos, CA (US); Erik G. de Jong, San Francisco, CA (US); Antonio F. Herrera, Woodside, CA (US); James G. Horiuchi, Sunnyvale, CA (US); Christopher J. Iacobucci, Ben Lomond, CA (US); Manish Mittal, Cupertino, CA (US)

(21) Appl. No.: 18/826,978

(22) Filed: Sep. 6, 2024

Related U.S. Application Data
(60) Provisional application No. 63/626,921, filed on Jan. 30, 2024.
(60) Provisional application No. 63/684,788, filed on Aug. 19, 2024.

(51) Int. Cl. G04G 17/06; G04G 17/04; G04G 17/08

(52) U.S. Cl. CPC G04G 17/06; G04G 17/045; G04G 17/08

(57) ABSTRACT

An electronic device can include a housing, a display assembly connected to the housing, a cover coupled to the housing opposite the display assembly, the housing being disposed between the cover and the display assembly. The cover can include a non-metallic portion including a perimeter edge, the non-metallic portion defining an external surface. Additionally, a metal portion can surround the non-metallic portion between the non-metallic portion and the housing. Additionally, an electrical conductor can extend from the external surface defined by the non-metallic portion and around the perimeter edge between the non-metallic portion and the metal portion, and an insulator can be disposed between the metal portion and the electrical conductor.

### CROSS-REFERENCE TO RELATED APPLICATIONS

This claims priority to U.S. Provisional Patent Application No. 63/684,788, filed 19 Aug. 2024, and entitled “ELECTRONIC DEVICE,” and U.S. Provisional Patent Application No. 63/626,921, filed 30 Jan. 2024, and entitled “ELECTRONIC DEVICE,” the entire disclosures of which are hereby incorporated by reference.

### FIELD

The present disclosure relates generally to electronic devices. More particularly, the present disclosure relates to wearable electronic devices.

### BACKGROUND

Electronic devices are increasingly being designed with device portability in mind. For example, devices are being designed to allow users to use these devices in a wide variety of situations and environments. In the context of wearable devices, these devices can be designed to include many different functionalities and to be operated in many different locations and environments. The components of an electronic device, such as processors, memory components, antennas, displays, and other components, can operate individually or together to perform those many functions. Thus, further tailoring and arranging components for electronic devices to provide additional or enhanced functionality without introducing or increasing undesirable device properties, such as size and weight, is desirable.

### SUMMARY

In at least one example of the present disclosure, an electronic device can include a housing, a display assembly connected to the housing, a cover coupled to the housing opposite the display assembly, the housing being disposed between the cover and the display assembly. The cover can include a non-metallic portion including a perimeter edge, the non-metallic portion defining an external surface. Additionally, a metal portion can surround the non-metallic portion between the non-metallic portion and the housing. Additionally, an electrical conductor can extend from the external surface defined by the non-metallic portion and around the perimeter edge between the non-metallic portion and the metal portion, and an insulator can be disposed between the metal portion and the electrical conductor.

In some examples of the electronic device, the housing defines an internal volume, and the electronic device further includes an electronic component disposed in the internal volume, an electrode disposed on the external surface defined by the non-metallic portion, and the electrical conductor can electrically couple the electrode to the electronic component. In other examples, the insulator includes a plastic insulator disposed between the metal portion and the perimeter edge. In yet other examples, the insulator includes an insulative physical vapor deposition (PVD) coating extending around the perimeter edge between the electrical conductor and the metal portion. In some examples the PVD coating includes at least one of silicon nitride, silicon oxynitride, or silicon dioxide. In some examples of the electronic device, the insulator defines the external surface. In other examples, the non-metallic portion includes a transparent window, which can be sapphire. In yet other examples, the electronic device further includes a light emitter disposed in an internal volume defined by the housing and configured to transmit light through the transparent window.

In at least one example of the present disclosure, an electronic display device includes a housing, a display assembly coupled to the housing, and a cover coupled to the housing opposite the display assembly. In some examples the cover includes, the cover includes a transparent window including a perimeter edge, the transparent window defining an external surface, and further includes a metal portion surrounding the transparent window. The electronic display device can further include an electrical conductor extending from the external surface defined by the transparent window and around the perimeter edge between the transparent window and the metal portion. Further, a plastic insulator can surround the transparent window between the metallic portion and the electrical conductor.

In some examples of the electronic display device, the plastic insulator extends around a perimeter of the transparent window. In other examples the plastic insulator defines the external surface. In some examples the electronic display device further includes a sensor assembly disposed within the housing, and an electrode disposed on the external surface defined by the transparent window, the electrode electrically coupled to the sensor assembly via the electrical conductor. In some examples the transparent window includes sapphire. In other examples of the electronic display device, the housing includes metal, the plastic insulator is a first plastic insulator, and the electronic display device includes a second plastic insulator disposed between the metal portion and the housing. The electronic display device can further include an antenna assembly including the metal portion.

In at least one example of the present disclosure, a display device includes a housing, a display assembly connected to the housing, and a cover connected to the housing opposite the display assembly. In some examples the cover includes a non-metallic portion including a perimeter edge, the non-metallic portion defining an external surface, and a metal portion disposed between the non-metallic portion and the housing. The display device can also include an electrical conductor extending from the external surface defined by the non-metallic portion and around the perimeter edge between the non-metallic portion and the metal portion, and an electrically insulating coating extending on the perimeter edge between the electrical conductor and the metal portion.

In some examples of the display device, the electrically insulating coating includes a ceramic material. In some other examples, the display device further includes an electrode disposed on the external surface of the non-metallic portion, the electrode in electrical communication with an electronic component within the display device via the electrical conductor. In other examples, the non-metallic portion includes a transparent window, and the display device further includes a light emitter configured to transmit light through the transparent window.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A shows a top perspective view of an example of a wearable electronic device;

FIG. 1B shows another top perspective view of the wearable electronic device;

FIG. 1C shows an exploded view of the wearable electronic device;

FIG. 1D shows a bottom perspective view of the wearable electronic device;

FIG. 1E shows a side view of the wearable electronic device;

FIG. 1F shows another side view of the wearable electronic device;

FIG. 1G shows another bottom perspective view of the wearable electronic device;

FIG. 2A shows an internal cutaway view of an example of a wearable electronic device;

FIG. 2B shows a cross-sectional side view the wearable electronic device shown in FIG. 2A;

FIG. 2C shows a zoomed-in view of a portion of the cross-sectional side view shown in FIG. 2B;

FIG. 2D shows a cross-sectional view of the wearable electronic device shown in FIG. 2A;

FIG. 3A illustrates a bottom perspective view of an example of a wearable electronic device;

FIG. 3B shows a cross-sectional view of a portion of an example of a wearable electronic device;

FIG. 4 shows a cross-sectional view of a portion of an example of a wearable electronic device;

FIG. 5A shows a cross-sectional view of a portion of an example of a wearable electronic device;

FIG. 5B shows a cross-sectional view of a portion of an example of a wearable electronic device;

FIG. 6 shows a cross-sectional view of a portion of an example of a wearable electronic device;

FIG. 7 shows a cross-sectional view of a portion of an example of a wearable electronic device;

FIG. 8A shows a plan view of a portion of an example of a wearable electronic device;

FIG. 8B shows a plan view of a portion of an example of a wearable electronic device;

FIG. 9A shows a perspective view of a portion of an example of a wearable electronic device;

FIG. 9B shows a cross-sectional perspective view of a portion of an example of a wearable electronic device;

FIG. 9C shows a perspective view of a portion of a portion of an example of a wearable electronic device;

FIG. 9D shows a bottom perspective view of a portion of an example of a wearable electronic device;

FIG. 10 shows a cross-sectional view of a portion of a portion of an example of a wearable electronic device;

FIG. 11 shows a plan view of a portion of a portion of an example of a wearable electronic device;

FIG. 12 shows a plan view of a portion of a portion of an example of a wearable electronic device;

FIG. 13 shows a cross-sectional view of a portion of a portion of an example of a wearable electronic device;

FIG. 13A shows a partial, perspective, cutaway view of a portion of an example of a wearable electronic device;

FIG. 14 shows a plan view of a portion of a portion of an example of a wearable electronic device;

FIG. 15 shows a plan view of a portion of a portion of an example of a wearable electronic device;

FIG. 16 shows a plan view of a portion of a portion of an example of a wearable electronic device;

FIG. 17 shows a cross-section view of a portion of a portion of an example of a wearable electronic device;

FIG. 18 shows a top plan view of an example of a wearable electronic device;

FIG. 19 shows a side view of a portion of an example of a wearable electronic device;

FIG. 19A shows a perspective view of a portion of an example of a wearable electronic device;

FIG. 19B shows a perspective view of a portion of an example of a wearable electronic device;

FIG. 20 shows a cross-sectional view of an example of a speaker module of a wearable electronic device;

FIG. 21 shows a perspective view of an example of a speaker module of a wearable electronic device;

FIG. 22 shows a perspective view of an example of a speaker module of a wearable electronic device;

FIG. 23 shows a cross-sectional view of an example of a speaker module of a wearable electronic device;

FIG. 24A shows a side view of an example of a wearable electronic device;

FIG. 24B shows a cross-sectional view of the wearable electronic device of FIG. 24A;

FIG. 24C shows a close-up view of the side view shown in FIG. 24A;

FIG. 25A shows a side view of a portion of an example of a wearable electronic device;

FIG. 25B shows a partial plan view of an example of a wearable electronic device;

FIG. 25C shows a perspective view of an example of a push-button of a wearable electronic device;

FIG. 25D shows a cross-sectional view of the portion shown in FIG. 25B;

FIG. 25E shows an example of a button retention clip; and

FIG. 26 shows a cross-sectional view of an example of a dial button of a wearable electronic device.

### DETAILED DESCRIPTION

Reference will now be made in detail to representative examples illustrated in the accompanying drawings. It should be understood that the following descriptions are not intended to limit the embodiments to one preferred example or embodiment. To the contrary, it is intended to cover alternatives, modifications, and equivalents as can be included within the spirit and scope of the described embodiments as defined by the appended claims.

The following disclosure generally relates to electronic devices. More particularly, the present disclosure relates to wearable electronic devices. The wearable electronic devices of the present disclosure include tailored arrangements of components to provide additional or enhanced functionality without introducing or increasing undesirable device properties, such as weight and size, and increasing performance. In this way, more functionality and componentry can be included in wearable devices for users to wear and operate in a variety of conditions and activities without limiting the functionality and durability of the devices.

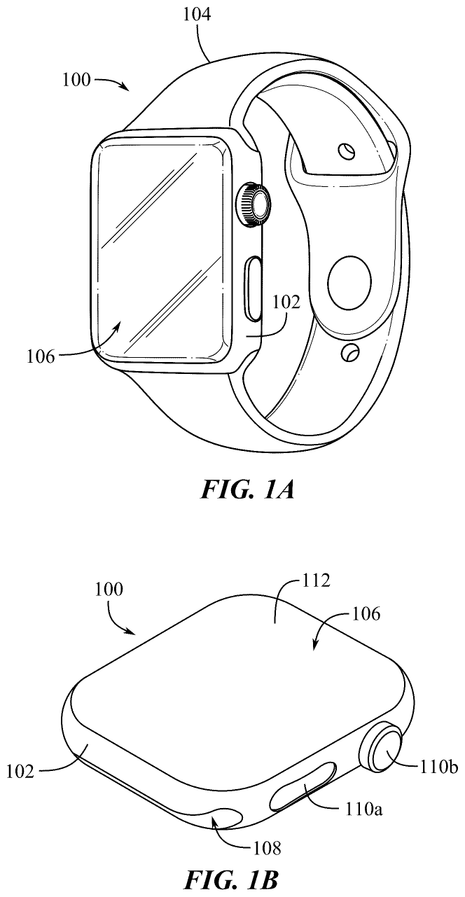

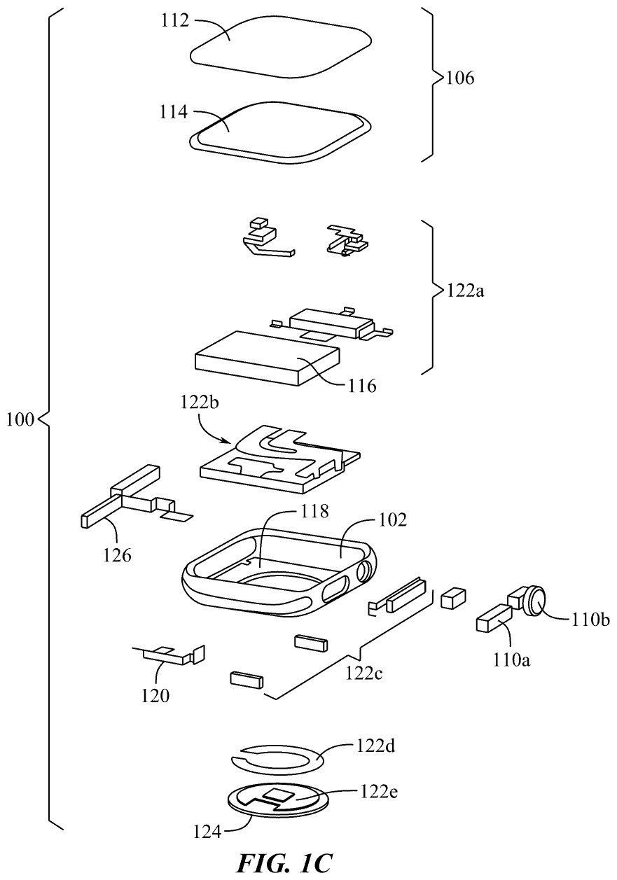

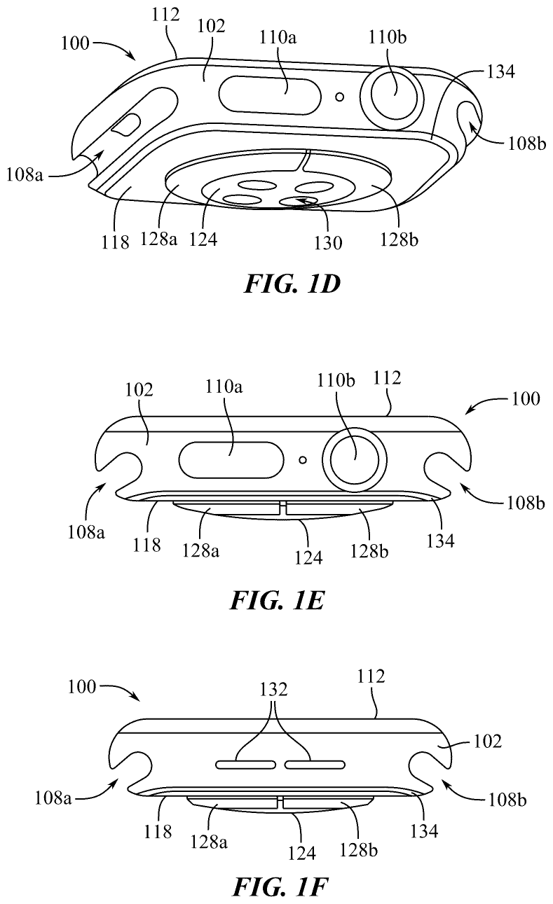

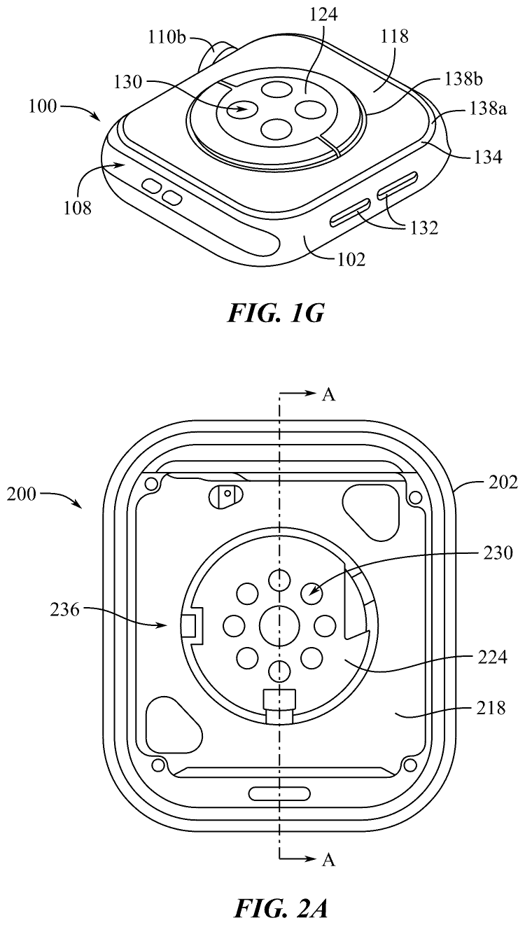

Specific examples and embodiments of electronic devices, including wearable electronic devices, are discussed below with reference to FIGS. 1-26. However, those skilled in the art will readily appreciate that the detailed description given herein with respect to these Figures is for explanatory purposes only and should not be construed as limiting. Furthermore, as used herein, a system, a method, an article, a component, a feature, or a sub-feature comprising at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

### Metal Rear Cover and Antenna Split

In at least one example of the present disclosure, a wearable electronic device can include a display assembly configured to face the user when the device is worn and a rear cover opposite the display assembly. A sidewall housing can be disposed between the display assembly and the rear cover. In at least one example, the rear cover can include a metal portion and be a part of an antenna assembly. The sidewall housing can also be metal or another conductive material and can be part of the antenna assembly. The antenna assembly can include a non-conductive antennal split disposed between and electrically isolating the metal rear cover from the conductive sidewall housing. In this way, the rear cover can be made of durable, stiff material to increase device strength and longevity while also resonating and/or radiating independently from the sidewall housing to send and receive signals as part of the antenna assembly. In at least one example, the metal rear cover defines an external surface of the device such that a distance between the antenna plane in which the rear cover is disposed and the antenna plane in which the sidewall housing is disposed is maximized for heightened antenna performance.

FIGS. 1A-1G show various views of an example of an electronic device 100 according to the present disclosure. FIG. 1A shows a perspective view of the electronic device 100. The electronic device shown in FIG. 1A is a watch, such as a smartwatch. The smartwatch of FIG. 1A is merely one representative example of a device that can be used in conjunction with the systems and methods disclosed herein. Electronic device 100 can correspond to any form of wearable electronic device, a portable media player, a head-mountable display, a media storage device, a portable digital assistant (“PDA”), a tablet computer, a computer, a mobile communication device, a GPS unit, a remote-control device, or other electronic device. The electronic device 100 can be referred to as an electronic device, or a consumer device. In some examples, the electronic device 100 can include a housing 102 that can carry operational components, for example, in an internal volume at least partially defined by the housing 102. The electronic device 100 can also include a securement strap including a securement band 104, or other retaining component that can secure the device 100 to a body of a user as desired.

In at least one example, the device 100 can include a display assembly 106 secured to the housing 102 or at least partially within the housing 102. The display assembly 106 can include one or more displays configured to project light to the user and provide a user-interface with which the user can interact. In at least one example, the display assembly 106 includes a touchscreen for receiving touch input from the user.

FIG. 1B illustrates a perspective view of the device 100 without the band 104 to illustrate a band gap 108 configured to receive and retain an end of the band 104 to the housing 102. In at least one example, the housing 102 can define the band gap 108 and the band 104 can be removably secured to the housing 102 at the band gap 108. The perspective view of FIG. 1B illustrates a single band gap 108 and the device 100 can include a second band gap (not shown) opposite the band gap 108 to receive an opposite end of the band 104.

In addition to the touch-input of the display assembly 106 noted above, one or more examples of the device 100 can include input mechanisms such as buttons 110a, 110b. The first button 110a can be disposed in a first aperture defined by the housing 102 and be depressible relative to the housing 102. The second button 110b can include a rotatable and/or depressible dial that the user can manipulate. The first and second buttons 110a-b are non-limiting examples of buttons of the device 100 and other examples can include more or less than the buttons 110a-b shown and located in various locations on or in the device 100. The buttons 110a-b are configured to be manipulated by the user to input commands resulting in changes in outputs of the display assembly 106 or other devices wirelessly or otherwise communicatively coupled with one or more processors, antennas, and so forth, of the device 100.

In at least one example, the display assembly 106 includes a transparent cover 112 defining an exterior surface of the device 100 configured to face the user when the user wears the device 100 via the band 104. In at least one example, the display assembly 106 includes one or more displays, for example display screens, positioned and configured to project light through the transparent cover 112 toward the user.

FIG. 1C illustrates an exploded view of the device 100 shown in FIG. 1B, showing various external and internal components. In at least one example, the device 100 can include the display assembly 106 having the transparent cover 112 and one or more displays 114. A battery 116 cab be disposed within the housing 102 and a rear cover 118 can be secured or disposed with/on the housing 102 opposite the display assembly 106. In one example, the battery 116 can be disposed between the display assembly 106 and the rear cover 118 when the device 100 is assembled. In at least one example, the rear cover 118 of the device 100 can also include a rear window 124 defining an external surface of the device 100, or at least one or more external surfaces or portions of many portions that form the entirety of the external surface of the device 100. The window 124 can include a material, or a combination of materials, that are transparent to light, for example transparent to infrared (IR) light. In at least one example, the window 124 can include a ceramic material, a glass material, a transparent polymer or material such as plastic, a crystal material, or other materials or combinations thereof. In one example, one or more emitters and sensors can be disposed within the device 100 and positioned/configured to emit and receive light, respectively, through the window 124.

FIG. 1C also shows the first and second buttons 110a, 110b also illustrated in FIG. 1B. In one example, the device 100 can include a pressure sensor module 126 configured to measure an atmospheric and/or internal pressure of the device 100. In at least one example, the device 100 can include an antenna 120 disposed within the housing 102. The antenna 120 can be a restoring antenna configured to restore a wireless signal upon re-booting or initially powering of the device 100 after shutdown. The device 100 can include various other electronic components 122a, 122b, 122c, 122d, 122e including one or more antennas, electrical cables including flat electrical cables, circuitry, processors, speakers, printed circuit boards and logic boards, memory components, haptic engines, light sources, and so forth.

FIGS. 1D-1F illustrate further views of the wearable electronic device 100, including a lower perspective view in FIG. 1D, a right-side view in FIG. 1E, a left side view in FIG. 1F, and a bottom perspective view in FIG. 1G. As illustrated, the device 100 includes the housing 102, the transparent cover 112 secured to the housing 102 opposite the rear cover 118 secured to the housing 102, the transparent window 124 secured to the rear cover 118, and the first and second buttons 110a-b. In at least one example, the device 100 includes first and second touch electrodes 128a, 128b on the transparent window 124. The various views of FIGS. 1D-1G also illustrate first and second band gaps 108a, 108b for receiving and securing to opposing ends of the securement band 104. The device 100 can also include one or more sensors and emitters of a sensor array 130 disposed within the device 100 and configured to send and receive signals through the transparent window 124. FIG. 1F illustrates one or more speaker ports 132 including apertures defined by the housing 102.

In at least one example, the device 100 can include a non-conductive split 134 disposed between the housing 102 and the rear cover 118. In one example, the split 134 can couple the rear cover 118 to the housing 102. In at least one example, the housing 102 includes a sidewall or sidewall housing extending between the display assembly 106 and the rear cover 118. In one example of the device 100, the rear cover 118 can include an electrically conductive material including one or more metal, conductive ceramic or glass, conductive composite material, and so forth. The electrically conductive rear cover 118 can define an external surface of the device 100 and can be coupled to one or more antenna circuitry components within the housing 102 and serve as a radiator of an antenna assembly. The housing 102 can also define the external surface.

In at least one example of the present disclosure, the sidewall housing 102 can include one or more electrically conductive materials such as metal and the rear cover 118 can be electrically isolated and insulated from the sidewall housing 102 via the non-conductive split 134, which provides a split or division between the conductive materials. In this way, the rear cover 118 can be a radiator of an antenna assembly and the non-conductive split 134 can be disposed between the metal housing 102 sidewall and the metal radiator of the rear cover 118 to electrically isolate the metal radiator of the rear cover 118 from the metal housing 102 sidewall. In such an example, the device 100 can include an antenna assembly, including one or more antenna circuitry 122a-e components and the metal radiator of the rear cover 118, which can be configured to radiate relative to the metal housing 102.

In at least one example, the non-conductive split 134 can include a plastic material defining the external/exterior surface. In at least one example, the non-conductive split 134 can include an epoxy material. In one example, the non-conductive split 134 can include a plastic material defining the external surface of the device 100 and an epoxy portion bonded or molded to the plastic and disposed interior to the housing 102, for example not defining the external surface and/or defining an internal volume within the device 100. In at least one example, the split 134 includes a first plastic portion 138a defining the external surface between the metal radiator (rear cover 218) and the metal sidewall housing 102 and a second plastic portion 138b defining the external surface between the transparent window 124 and the rear cover 118.

Any of the features, components, and/or parts, including the arrangements and configurations thereof shown in FIGS. 1A-1G can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 1A-1G.

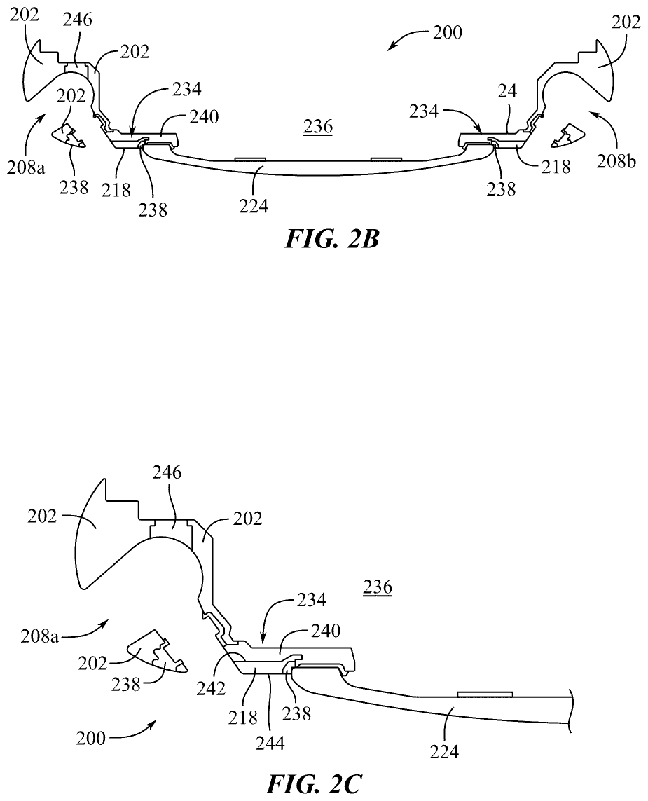

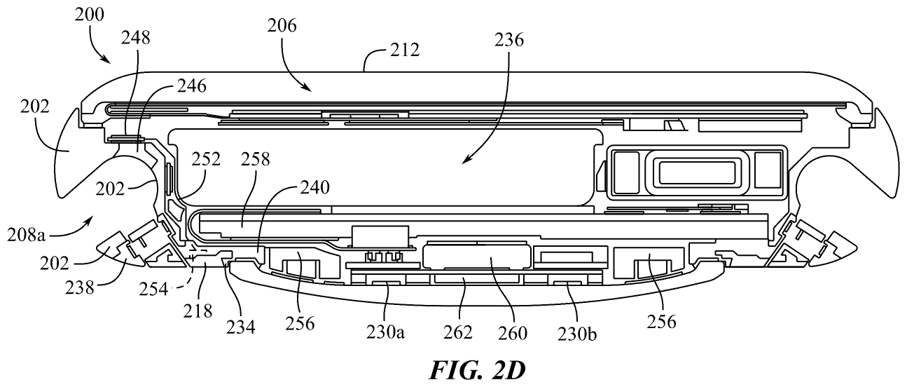

FIGS. 2A-2D illustrate various views of a wearable electronic device 200, which can be similar to the device 100 shown in FIGS. 1A-1G. FIG. 2A illustrates a top, plan view of the device 200 without a front cover or display assembly and without various internal components such that an internal surface of the rear cover 218, which can include the transparent window 224, is visible. As shown, the rear cover 218 can define an internal volume 236 of the device 200. FIG. 2A also illustrates a sensor assembly 230 aligned with the transparent window 224, which can include one or more light barriers to guide light in and out through the window 224 as well as various emitters, receivers, and so forth. The housing 202 also defines the internal volume 236.

FIG. 2B illustrates a cross-sectional view of the device 200 shown in FIG. 2A along the plane A-A indicated in FIG. 2A and FIG. 2C illustrates an enlarge view of a portion thereof. The device 200 can include the housing 202 coupled to the rear cover 218 via the non-conductive split 234 and the window 224 as a part of, or coupled with, the rear cover 218. In addition, in at least one example, the housing 202 can define first and second band slots 208a, 208b.

In at least one example, the split 234 includes a plastic portion 238 defining an external surface of the device 200 and an epoxy portion 240 defining the internal volume 236. In at least one example, the epoxy portion 240 and the plastic portion 238 of the split 234 can be molded or bonded together, for example in a two-shot molding process. As noted above, the rear cover 218 can be a metal radiator as part of an antenna assembly of the device 200. In at least one example, the plastic portion 238 defines the external surface, or at least one portion of the external surface of the device 200 between the metal radiator (rear cover 218) and the sidewall housing 202. In at least one example, the epoxy portion 240 of the split 234 can be coupled to both the first and second plastic portions (e.g., 138a and 138b shown in FIG. 1G) of the split 234. In at least one example, the epoxy portion 240 is bonded to an internal surface 242 of the metal radiator (e.g., the metal rear cover 218) opposite the external surface 244 thereof.

In at least one example, as shown in at least FIGS. 2B and 2C, the housing 202 of the device 200 can include or define a radio-frequency (RF) window 246 defining the band slot 208a. In at least one example, the RF window 246 includes a non-conductive, RF-transparent material configured to allow RF signals to cross from the internal volume 236 to the external environment through the metal sidewall housing 202. As shown in the cross-sectional view of the device 200 in FIG. 2D, an antenna resonator 248 can be coupled to an internal surface of the RF window 246 within the internal volume 236. In one example, the antenna resonator 248 can be electrically coupled to an antenna feed point 254 electrically coupled or in contact with the internal surface of the metal radiator rear cover 218 (e.g., at the internal surface 242 of the rear cover 218 illustrated and labeled in FIG. 2C). The antenna resonator 248 can be electrically coupled to the antenna feed point 254 via a conductive cord 252 extending from the antenna feed point 254 to the antenna resonator 248 at, near, or adjacent the RF window 246. In this way, in at least one example, the antenna resonator 248 can resonate with the rear cover 218 acting as an antenna radiator, to restore a wireless signal upon a resetting, rebooting, or start-up of the electronic device 200.

In at least one example, the conductive cord 252 can include a flat electrical cord or cable, for example a cord or cable having a width five times greater than a height thereof. In at least one example, a flat electrical cable of the conductive cord 252 can be a flexible printed circuit (FCP), for example a flat FCP. The FCP can include a flexible, foldable printed circuit having one or more layers of copper or other conductive material traces.

In at least one example, the transparent cover 212 of the display assembly 206 can be a front cover disposed opposite a back cover including the rear cover 218 and the window 224. The housing 202 can be a sidewall housing including a metal material extending between the front cover and the back/rear cover 218.

In at least one example, the device 200 can include a charging coil 256 disposed in the internal volume 236 at, near, or adjacent the window 224. The epoxy portion 240 of the split 234 can be disposed between the metal radiator (e.g., the rear cover 218) and the charging coil 256 and be configured to electrically isolate the conductive rear cover 218 from the charging coil 256. In addition, in at least one example, the device 200 can include a printed circuit board (PCB) 258 disposed in the internal volume 236 at, near, or adjacent the window 224. The epoxy portion 240 of the split 234 can be disposed between the metal radiator (e.g., the rear cover 218) and the PCB 258 and be configured to electrically isolate the conductive rear cover 218 from the PCB 258.

In at least one example, the device 200 can include a magnet 260 centrally located at, near, or adjacent the PCB 258 and/or the window 224. The magnet 260 can be centrally located with the PCB 258 and one or more other electronic components, including various sensors 230a, 230b of a sensor array configured to emit and receive signals through the window 224, disposed peripherally around the magnet 260. In at least one example, the magnet 260 can be configured to magnetically secure the device 200 to a charging station or component. The single, centrally locate magnet 260 can provide a larger force-per-thickness of the magnet to reduce the overall thickness of the device 200. The magnet 260 can be disposed between the PCB 258 and the window 224. In addition, in at least one example, the device 200 can include a flux director 262, which can include a carbon and/or steel layer disposed between the magnet 260 and the window 224. The flux director 262 can be positioned and configured to direct flux from the magnet 260 out through the window 224 for intentional directional attraction to external devices such as magnetic chargers, stands, cases, and the like.

Any of the features, components, and/or parts, including the arrangements and configurations thereof shown in FIGS. 2A-2D can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 2A-2D.

### Back-Dome and Electrode Isolation

In at least one example of the present disclosure, a wearable electronic device can include a rear cover configured to press against a body of the user, for example a wrist, when the device is worn. The rear cover can include a metallic portion and a non-metallic portion, for example a transparent sensor window formed of glass, plastic, or the like, surrounded by a metal portion. In some examples, external sensors such as touch sensitive electrodes can be placed on the external surface of the non-conductive window to provide input and data from the wearer. Due to the conductive nature of the metal portion of the rear cover surrounding the window, the device can also include one or more insulators disposed between and electrically isolating the electrodes from the metal portion of the rear cover. In this way, the metal portion of the rear cover can provide a durable, stiff housing to the device and the electrodes interacting with the user's body can function without interference or electrical cross-talk from the adjacent metal rear cover.

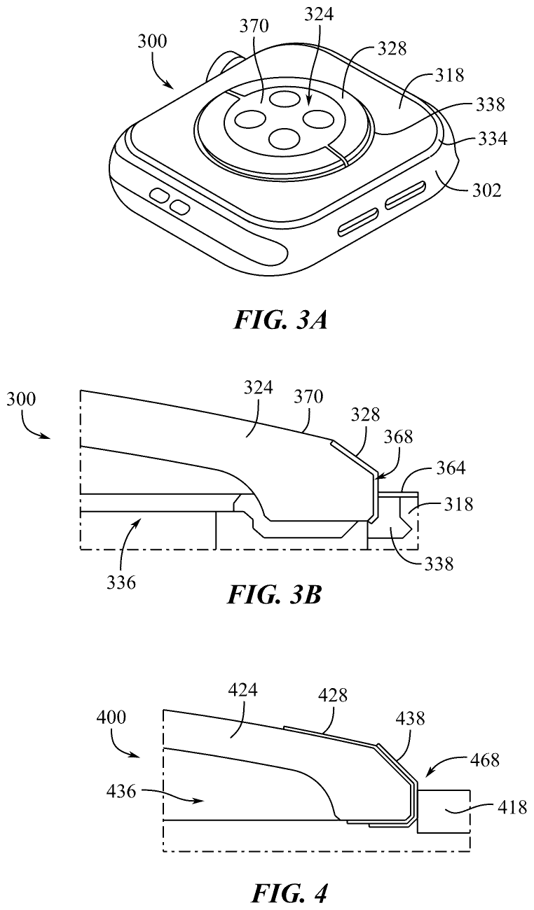

FIGS. 3A illustrates a bottom perspective view of an example of a wearable electronic device 300 including a sidewall housing 302 and a rear cover 318 including a transparent window 324. The transparent window 324 can include an IR-transparent material. In at least one example, the rear cover 318 includes a metal or otherwise electrically conductive material such that the rear cover 318 can function as an antenna radiator as part of an antenna assembly of the device 300. In at least one example, the device 300 can also include one or more electrical conductors 328, which can include touch sensitive electrodes for receiving touch input from a user, disposed on an external surface of the window 324. In the illustrated example of FIG. 3A, the electrical conductor 328 extends at least partially around a perimeter region or edge of the window 324. Other examples can include other electrical conductors and electrodes in different positions or on different regions of the window 324.

In at least one example, the device 300 can include an insulator 338 (e.g., similar to the second plastic portion 138b of the non-conductive split 234 shown in FIG. 1G) disposed between the window 324 and the rear cover 318. The device 300 can also include a non-conductive split 334 between the metal sidewall housing 302 and the metal rear cover 318. In at least one example, insulator 338 is configured to electrically isolate the metal rear cover 318 from the electrical conductor 328 extending on or around a perimeter edge of the window 324 between the window 324 and the insulator 338.

For example, FIG. 3B shows a close-up, cross-sectional view of the perimeter edge 368 of the window 324 adjacent or near the insulator 338, which is disposed between the electrical conductor 328 and the metal rear cover 318 to electrically isolate the metal rear cover 318 from the electrical conductor 328. In at least one example, the rear cover 318 can be a metallic portion of the device 300 at least partially surrounding the window 324. The window 324 can be a non-metallic and otherwise non-conductive component or portion of the device 300 defining an external surface 370 of the device 300, or at least one portion of many portions of the external surface. In one example, the window 324 can include glass, ceramic, sapphire, or the like. In at least one example, as shown in FIG. 3B, the device 300 can also include a non-conductive coating 364 over the rear cover 318 and the insulator 338. The coating 364 can be a protective, scratch resistant coating to improve durability.

In at least one example, the electrical conductor 328 can extend around the perimeter edge 368 of the window 324, for example from an external surface 370 of the window 324, around the perimeter edge 368, and into the internal volume 336 of the device 336. In at least one example, the electrical conductor 328 can include a touch-sensitive electrode for detecting a user touch input. In such an example, the electrical conductor 328 can be electrically coupled to one or more electronic components within the device 300 as described herein. In at least one example, the electrical conductor 328 can extend from an electrode disposed on the external surface 370 defined by the window 324 to one or more electronic components disposed in the internal volume 336, for example one or more sensors or sensor assemblies.

Any of the features, components, and/or parts, including the arrangements and configurations thereof shown in FIGS. 3A-3B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 3A-3B.

FIG. 4 shows another example of a portion of an electronic device 400 including a window 424 adjacent a metal rear cover 418 and an electrical conductor 428 extending around a perimeter edge 468 of the window 424 between the window 424 and the rear cover 418 and into the internal volume 436. In the illustrated example, the device 400 includes an insulating coating 438 between the electrical conductor 428 and the metal rear cover 418. In this way, the coating 438 can electrically isolate the metal rear cover 418 from the electrical conductor 428. The coating 438 can define at least a portion of the external surface 470 of the device 400 and at least a portion of the electrical conductor 428 can define at least a portion of the external surface 470.

In at least one example, the coating 438 can be a physical vapor deposition (PVD) coating extending around the perimeter edge 468 of the window 424 between the electrical conductor 428 and the metal portion of the rear cover 418. In one or more examples, the PVD coating 438 can include at least one of silicon oxide, silicon oxynitride, or silicon dioxide. In at least one example, the coating 438 can include a ceramic material.

Any of the features, components, and/or parts, including the arrangements and configurations thereof shown in FIG. 4 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 4.

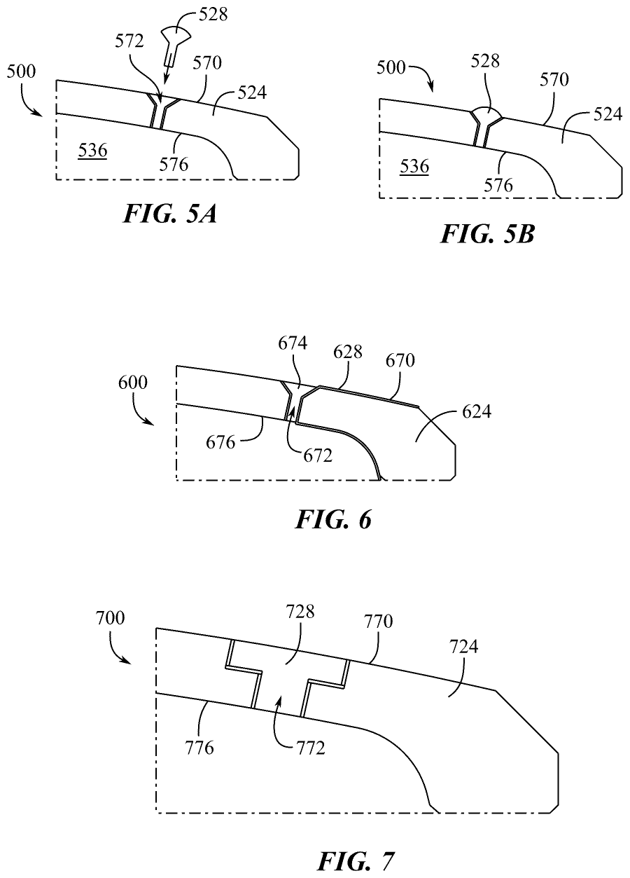

FIGS. 5A-5B illustrate a partial, cross-sectional view of another example of a device 500 that can include similar components and systems of other devices described herein. In the illustrated example of FIGS. 5A and 5B, the device 500 can include a window 524, for example a transparent or semi-transparent window of a rear cover of the device 500, defining an aperture 572 extending through the thickness of the window 524 between the external surface 570 and the internal surface 576 defined by the window 524.

In at least one example, an electrical conductor 528 can include a pin insert configured to be set or disposed within the aperture 572 such that when the pin insert is disposed in the aperture 572, as shown in FIG. 5B, the electrical conductor 528 extends through the thickness of the window 524 and defines both the external surface 570, or at least a portion of the external surface 570, and the internal surface 576 of the window 524. In this way, the electrical conductor 528 in the aperture 572 can act as an electrical pathway through the window 524, for example from an external electrode to an internal component within the internal volume 526 of the device 500.

Any of the features, components, and/or parts, including the arrangements and configurations thereof shown in FIGS. 5A-5B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 5A-5B.

FIG. 6 shows a partial, cross-sectional view of another example of a device 600 that can include similar components and systems of other devices described herein. In the illustrated example of FIG. 6, the device 600 can include a window 624, for example a transparent or semi-transparent window of a rear cover of the device 600, defining an aperture 672 extending through the thickness of the window 624 between the external surface 670 and the internal surface 676 defined by the window 624. In the illustrated example of FIG. 6, a fill material 674 can extend through the window 624 and an electrical conductor 628 can extend through the window between the fill material 674 and an inner surface of the window 624 defining the aperture 672 in which the fill material 674 is disposed. The electrical conductor 628 can extend through the window 624 to define an external surface 670 and an internal surface 676 or portions thereof.

Any of the features, components, and/or parts, including the arrangements and configurations thereof shown in FIG. 6 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 6.

FIG. 7 shows a partial, cross-sectional view of another example of a device 700 that can include similar components and systems of other devices described herein. In the illustrated example of FIG. 7, the device 700 can include a window 724, for example a transparent or semi-transparent window of a rear cover of the device 700, defining an aperture 772 extending through the thickness of the window 724 between the external surface 770 and the internal surface 776 defined by the window 724. In at least one example, the aperture 772 can include a counter-bore through-hole extending through the window 724 and the electrical conductor 728 disposed in the aperture 772 can be co-finished with the window 724 to form a flush, continuous external surface 770. The electrical conductor 728 can also define or extend to the internal surface 776 of the window 724. The electrical conductor 728 can be formed of one or more electrically conductive materials including metal to form an electrical pathway through the window 724, for example an electrical pathway between an external electrode and an internal electronic component, both of which can be electrically coupled via the electrical conductor 728.

Any of the features, components, and/or parts, including the arrangements and configurations thereof shown in FIG. 7 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 7.

### Wireless Restore Antenna Flex and Routing

In at least one example of the present disclosure, a wearable electronic device can include an antenna assembly having a metal rear cover separated from a conductive sidewall housing by a non-conductive antenna split, as well as an internal conductor electrically coupling the metal rear cover to an antenna resonator disposed within the device and against the sidewall housing. The sidewall housing can include a radio-frequency (RF) window at which the resonator is coupled such that the resonator is configured to resonate with the metal rear cover rather than the sidewall housing, even as the resonator is disposed adjacent or level with the sidewall housing. In this way, in examples where the metal rear cover is configured to resonate relative to the sidewall housing as part of an antenna assembly, as described elsewhere herein, the conductor and resonator disposed at the RF window of the sidewall housing can be optimally positioned to function as a wireless signal restoring antenna.

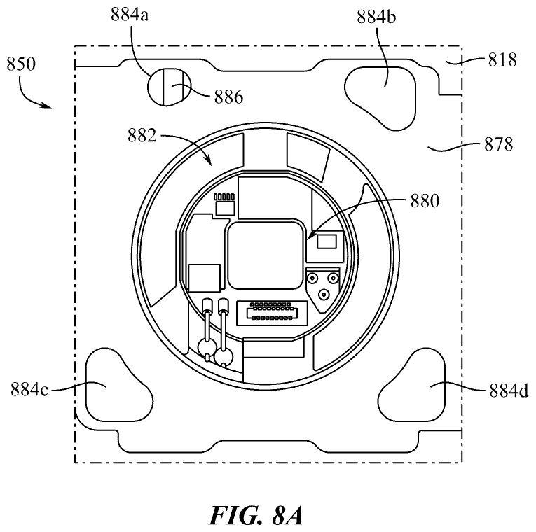

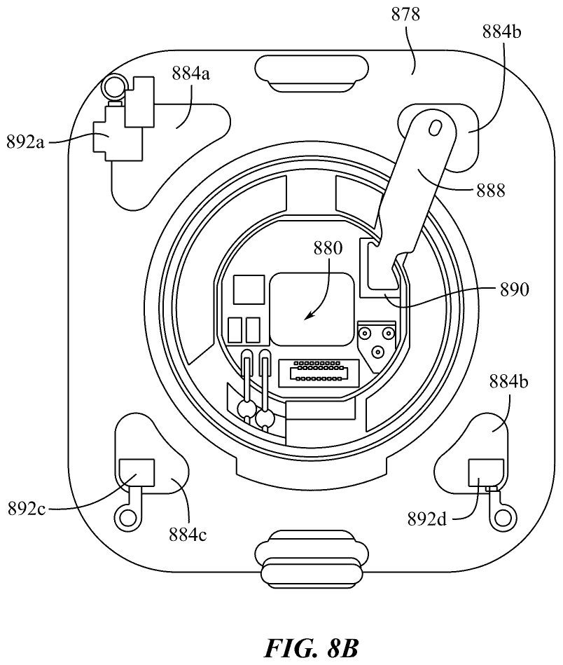

FIGS. 8A-8B show a top, plan view of a portion of a wearable electronic device 800, including an inner surface of a rear cover 818 defining an internal volume of the device 800. In at least one example, the device 800 can include a non-conductive portion 878 disposed against an internal surface of the metal rear cover 818. In at least one example, the non-conductive portion 878 can be an inlay shaped complimentary to the rear cover 818. In at least one example, the non-conductive portion can be formed of plastic, epoxy, or a combination thereof. In at least one example, the non-conductive portion 878 can connect to or form at least a portion of the insulator 338 and/or the non-conductive split 334 shown in FIG. 3A.

In at least one example, the non-conductive portion 878 can define a central aperture 882 aligned with the transparent window coupled to the rear cover 818 of the device 800. In at least one example, the device 800 can include one or more electronic components 880 located within or aligned with the aperture 882. The electronic components 880 can include one or more of a printed circuit board (PCB), integrated circuit including one or more processors, memory components, and so forth, one or more sensors of a sensor array, one or more antenna components including antenna feed points, resonators, radiators, and so forth, or other electronic components. In at least one example, the device 800 can include one or more apertures or through-holes through which one or more corresponding antenna feed points, including interposers 884a, 884b, 884c, 884d, make electrical contact with the metal rear cover 818. In at least one example, one or more of the metal interposers 884a-d can be welded to an internal surface 842 of the rear cover 818.

In at least one example, one or more of the interposers 884a-d can include stamped metal. In at least one example, one or more of the interposers 884a-d can include stainless steel. In at least one example, one or more of the interposers 884a-d can include gold plating 886 or a gold-plated region. In at least one example, as shown in FIG. 8B, the electronic device 800 can include a conductor 888 electrically coupling at least one interposer 884b with at least one of the electronic components 880 for example a PCB 890. In one example, the PCB 890 can include an antenna resonator as a part of the antenna assembly including the metal rear cover 818.

In at least one example, the conductor 888 can include a thin cable having a thickness/height less a width thereof, for example a thickness/height at least five-times less than the width. In at least one example, the thickness/height of the thin cable conductor 888 can be less than about 100 microns, for example less than about 75 microns, also for example about 50 microns or less. The thin electrical cable conductor 888 can reduce a vertical distance to maximize a distance between the average plane of the rear cover 818 resonance as part of the antenna assembly, thus improving antenna performance.

In at least one example, the first, third, and fourth interposers 884a, 884c, and 884d can serve as antenna ground points, with one or more electrical clips, springs, and/or fingers 892a, 892c, and 892d electrically coupled to an electrical ground and electrically coupled to the metal rear cover 818 of the device 800.

Any of the features, components, and/or parts, including the arrangements and configurations thereof shown in FIGS. 8A-8B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 8A-8B.

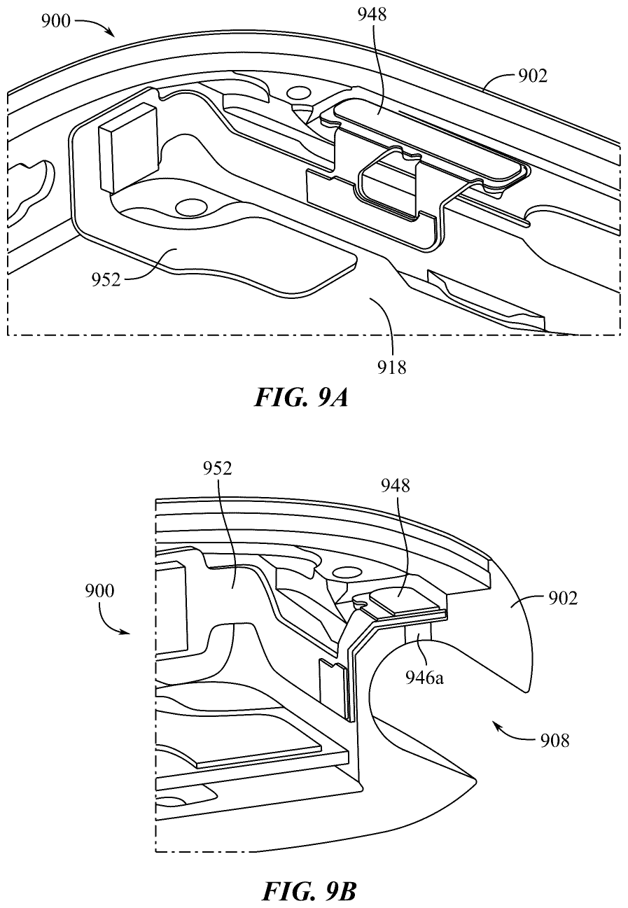

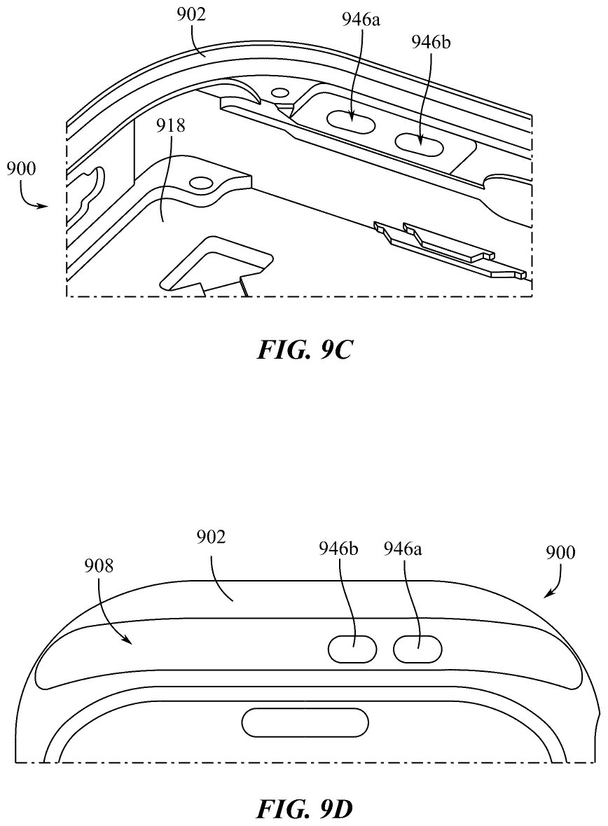

FIGS. 9A-9D show various views of a portion of an example of an electronic device 900. FIG. 9A shows a perspective view of various internal components within a sidewall housing 902 with a front cover and/or display assembly removed to show the internal components. In at least one example, the device 900 includes a metal rear cover 918 electrically isolated from and configured to resonate relative to, the metal sidewall housing 902. In at least one example, the device 900 includes a conductor 952, for example a flat conductive cord, electrically coupling the metal rear cover 918 to an antenna resonator 948. In at least one example, the conductive cord 252 can include a flat electrical cord or cable, for example a cord or cable having a width five times greater than a height thereof. In at least one example, a flat electrical cable of the conductive cord 252 can be a flexible printed circuit (FCP), for example a flat FCP. The FCP can include a flexible, foldable printed circuit having one or more layers of copper or other conductive material traces.

The antenna resonator 948 can thus be electrically coupled to the rear cover 918 such that the resonator 948 is configured to resonate with the metal rear cover 918 relative to the sidewall housing 902.

FIG. 9B shows a cutaway view of the device 900 with the cutaway planed passing through the resonator 948. In at least one example, the sidewall housing 902 defines a band slot 908 configured to receive and secure a securement band of the device 900 to the sidewall housing 902. As shown, the resonator 948 can be disposed at, near, or adjacent the band slot 908 on the inside of the device 900.

FIG. 9C shows another perspective view of the device 900 without the resonator 948 or conductor 952 shown. FIG. 9C shows first and second radio-frequency (RF) windows 946a and 946b extending through the housing 902. The RF windows 946a-b can be similar to the RF window 246 of the device 200 shown in FIG. 2D. In at least one example, the RF windows 946a-b extend through a thickness of the housing 902, as shown in the cutaway view of FIG. 9B. FIG. 9C illustrates the device 900 having two RF windows 946a-b. Other examples can include one or more than two RF windows located at various places on and through the housing 902.

In at least one example, the RF windows 946a-b can be transparent to RF signals sent and received by the resonator 948, which can be coupled to and/or disposed against, at, near, or adjacent the RF windows 946a-b. In this way, the signals sent and received by the resonator 948 can pass through the housing 902 as the resonator 948 resonates with the rear cover 918. The resonator 948 can resonate with the rear cover 918 via the conductor 952, as described above, even though the resonator 948 is disposed nearer to the sidewall housing 902 relative to which the rear cover 918 (and thus the resonator 948) resonates as part of the antenna assembly, because the resonator 948 is electrically isolated from the housing 902. In one example, the resonator 948 can be electrically isolated from the housing 902 at least in part by the RF windows 946a-b. In at least one example, the resonator 948 can be electrically isolated from the housing 902 at least in part by an intermediate insulating/non-conductive layer between the resonator 948 and the housing 902. Such a layer can include a non-conductive epoxy or other adhesion material coupling the resonator 948 to the housing 902 and/or to one or more of the RF windows 948a-b.

In at least one example, one or more of the RF windows 924a-b can extend through a thickness of the housing 902 to define the band slot 908, as shown in the cutaway view of FIG. 9B and the exterior, perspective view of FIG. 9D. In at least one example, one or more of the RF windows 948a-b can include epoxy. In at least one example, the conductor 952 can be electrically coupled to the metal rear cover 918 via an interposer, for example one of the interposers 884a-d shown in FIG. 8B and described above.

In at least one example, the resonator 948 can be configured to send and/or receive a wireless restoring signal upon re-booting or initially powering of the device 100 after shutdown. One or more external features of the housing 902 can be configured to direct an external wireless restore signal, for example electromagnetic waves, along one or more external surface or contours of the housing 902, for example a contour or external surface feature defining the band slot 908, toward one or more of the RF windows 946a-b and through to the resonator 948. In this way, the rear cover 918, conductor 952, resonator 948, one or more of the RF windows 946a-b, the housing 902, and the band slot 908 defined by the housing 902, can function as a wireless signal restoring antenna assembly.

Any of the features, components, and/or parts, including the arrangements and configurations thereof shown in FIGS. 9A-9B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 9A-9B.

### Display and Front-Crystal

In at least one example of the present disclosure, a wearable electronic device can include a display assembly having a transparent cover defining an external surface of the device.

In at least one example, the transparent cover can extend toward a peripheral edge of the device and define a convex, internal volume in which one or more display assembly components can be disclosed. This convex, internal geometry can provide additional volume and space for electronic components to function in a tightly packed, overall thin device, while maximizing the visible surface area of the display assembly when the user dons the device.

In at least one example of the present disclosure, a wearable electronic device can include a display assembly, a battery disposed within the device, and one or more electronic chips, for example a printed circuit board (PCB) having one or more sensors thereon, including an ambient light sensor (ALS), temperature sensor, and so forth. In some examples, the chip can be disposed between the battery and the chip and at least a portion of the chip can be free of electrical pins and solder. For example, at least one side or edge of the chip can be free of solder in order to reduce a space necessary to accommodate the chip between the battery and the display assembly. The chip can include one or more electrical vias to connect the side/edge disposed between the battery and display assembly, or any pins or traces thereof, to one or more other ground pins and solders disposed on other sides and edges of the chip. In this way, the chip can maintain the necessary electrical grounding while functioning during operation while being reduced in thickness and size to more closely disposed the battery relative to the display assembly. This can reduce the overall thickness of the device, which is desirable to customers wearing the device, without negatively impacting any functionality of the device.

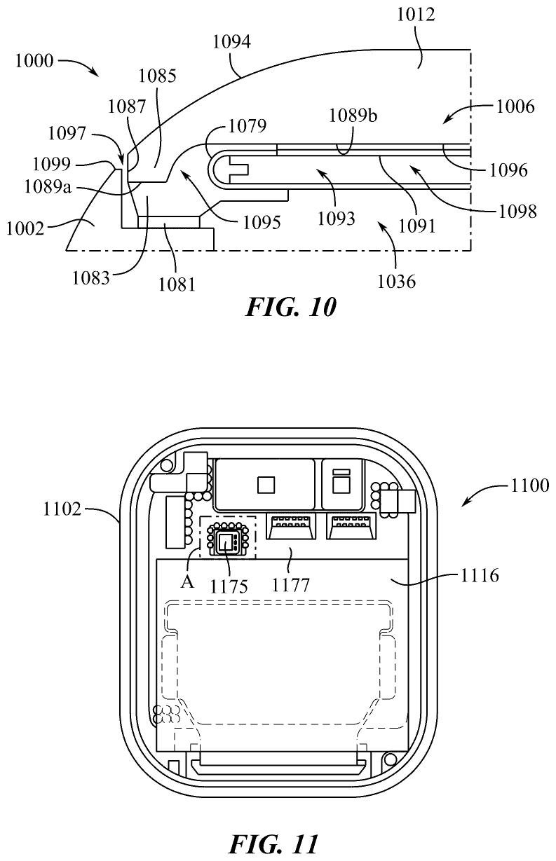

FIG. 10 shows a cross-sectional view of a portion of an example of a wearable electronic device 1000, including a display assembly 1006 coupled to a sidewall housing 1002. In at least one example, the sidewall housing 1002 can define an upper perimeter edge 1099. The display assembly 1006 can include a transparent cover 1012 defining an external surface 1094 of the device 1000 or at least a portion of the external surface 1094. The transparent cover 1012 can also include or define an internal surface 1096 thereof. In at least one example, the display assembly 1006 can include one or more display layers 1098. The display layers 1098 can include one or more displays, including display screens, and one or more display films. The display assembly 1006 can be coupled to the housing 1002 to define an internal volume 1036 of the wearable electronic device 1000.

In at least one example, the display assembly 1006 is coupled to the housing 1002 such that the transparent cover 1012 is separated from the upper perimeter edge 1099 of the housing 1002 by a gap 1097. The inner surface 1096 of the transparent cover 1012 can include a concave surface or portion 1095 defining a concave volume 1093 in which at least a portion of the display layers 1098 are disposed within the internal volume 1036 of the device 1000. In at least one example, the display layers 1098 include a display 1091, for example a display screen, disposed in the concave volume 1093. In at least one example, the display 1091 can include a curved outer edge portion 1079 disposed within the concave volume 1093. The concave surface portion 1095 of the internal surface 1096 of the transparent cover 1012 can form a first lower surface 1089a of the internal surface 1096 offset from a second lower surface 1089b of the internal surface 1096 to form or define the concave volume 1093.

In at least one example, the concave surface portion 1095 is curved. In other examples, the concave surface portion 1095 can include abrupt changes in slope of the internal surface 1096, for example one or more flat surfaces angled relative to one another, to form the concave volume 1093. In at least one example, the transparent cover 1012 includes an outer lip 1087 defining the first lower surface 1089a, the external surface 1094, an outer perimeter edge 1085 of the transparent cover 1012, and the concave surface portion. In at least one example, the outer perimeter edge 1085 is disposed adjacent the upper perimeter edge 1089 of the housing 1002. In at least one example, the outer perimeter edge 1085 is separated from the upper perimeter edge 1089 of the housing 1002 by the gap 1097, which can be an air gap or open gap with nothing disposed between the outer perimeter edge 1085 and the upper perimeter edge 1089.

In at least one example, the device 1000 includes a polymer material 1083 disposed between the outer lip 1087 and the display 1091. In at least one example, the polymer material 1083 is disposed between the transparent cover 1012 and the sidewall housing 1002. In at least one example, the polymer material 1083 is coupled to the sidewall housing 1002 via an adhesion layer 1081. The position of the various components of the assemblies shown in FIG. 10 enable a tight, thin stack-up for reducing an overall thickness of the wearable device 1000.

Any of the features, components, and/or parts, including the arrangements and configurations thereof shown in FIG. 10 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 10.

FIG. 11 shows an internal plan view of an example of a wearable electronic device 1100, including a battery 1116 disposed within a housing 1102. The device 1100 can also include a printed circuit board (PCB) 1177. In at least one example, an integrated circuit or chip 1175 can be electrically coupled to the PCB 1177. In one example, the chip 1175 can include an ambient light sensor and be electrically coupled to a processor/controller, for example via the PCB 1177, to control a brightness of the display of the device 1100 based on a measured ambient light. The major plane of the device 1100 can lie in the viewing plane of the plan view of FIG. 11. From this view, the battery 1116 can at least partially overlap the chip 1175. The battery 1116 can at least partially overlap and/or be stacked above chip 1175 in order to enable compact packaging to reduce an overall size of the device 1100 and to increase battery 1116 size and capacity.

Any of the features, components, and/or parts, including the arrangements and configurations thereof shown in FIG. 11 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 11.

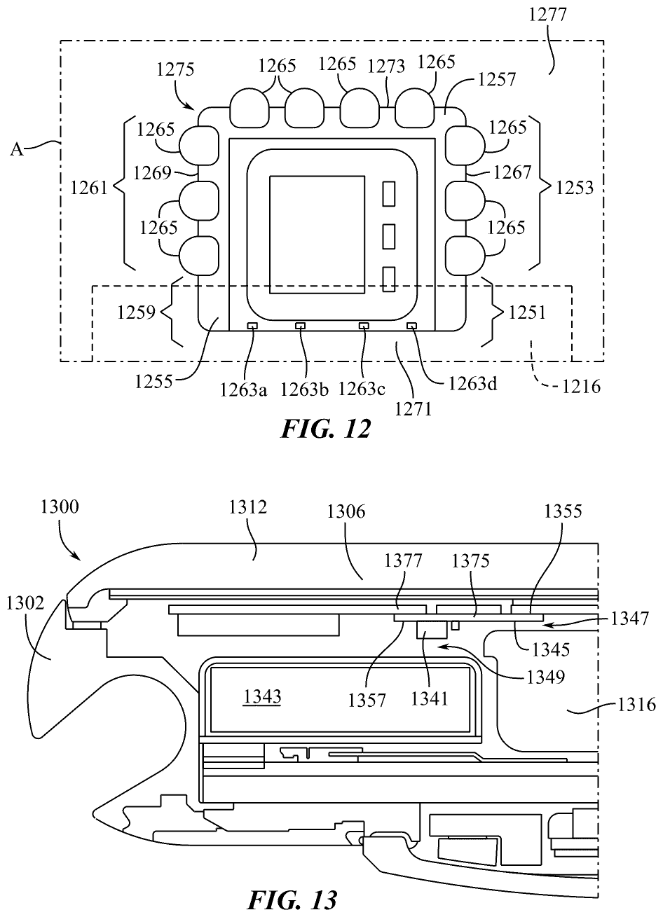

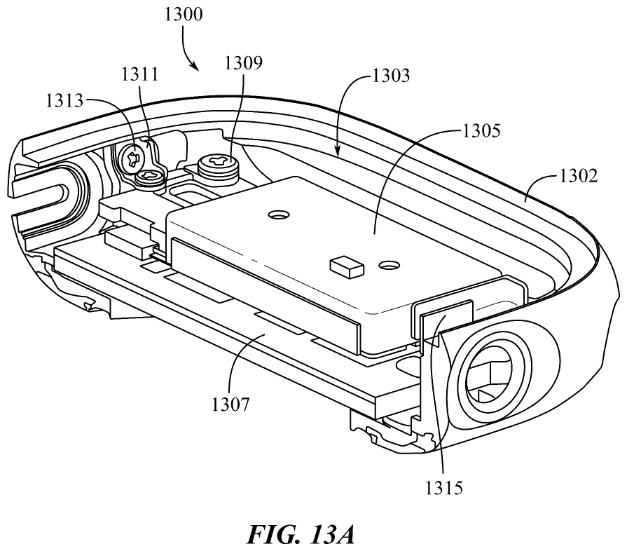

FIG. 12 shows a close-up view of an example PCB 1277 and battery 1216 similar to those seen in FIG. 11 in the area A indicated in FIG. 11. As shown in FIG. 12, the chip 1275 can be electrically coupled to the PCB 1277 via one or more electrical pins and/or solder points, which are described in more detail below. The chip 1275 can include a rectangular board having a first edge 1273 opposite a second edge 1271 and a third edge 1269 opposite a fourth edge 1267, with the third and fourth edges 1269, 1267 extending between the first and second edges 1273, 1271 and vice versa. As noted above, the chip 1275 can include a set of electrical pins 1265 electrically coupling the electronic chip 1275 to the PCB 1277 at the first edge 1273, the third edge 1269, and the fourth edge 1267. The second edge 1271 can be free of pins and/or solder points electrically coupling the chip 1275 to the PCB 1277. The second edge 1271 can be disposed underneath the battery 1216, in other words between the battery 1216 and the display assembly of the device 1200, as will be described below and shown in FIG. 13.

Referring still to FIG. 12, the chip 1275 can include one or more electrical vias 1263a-d electrically connecting a ground contact at or near the second edge 1271 to at least one electrical pin of the set of electrical pins 1265. In at least one example, the third edge 1269 can include a first length 1261 defining a first portion 1257 of the chip 1275 and a second length 1259 defining a second portion 1255 of the chip 1275. At least one of the electrical pins 1265 can be disposed along the first length 1261 and the second length 1259 can be disposed between the battery 1216 and the display assembly as shown in FIG. 12 where the battery 1216 indicated in dotted lines would overlap the second length 1259 and portion 1255 of the chip 1275. In at least one example, the second portion 1255 and second length 1259 of the third edge 1269 can be free of electrical pins.

In at least one example, the fourth edge 1267 includes a first length 1253 defining the first portion 1257 and a second length 1251 defining the second portion 1255. At least one of the electrical pins 1265 is disposed along the first length 1253 and no pins are disposed along the second length 1251 between the battery 1216 and the display. The various pins 1265 illustrated can be soldered to the PCB 1277 such that the pins 1265 are soldered to make an electrical connection between the chip 1275 and the PCB 1277. Any one or more of the pins 1265 can include an electrical grounding pin to electrically ground the chip 1275 to the PCB 1277.

In at least one example, the second edge 1271 is free of solder points and/or pins 1265 to allow for closer stacking between the battery 1216 and the chip 1275. In order to maintain a Faraday cage to electrical isolate and protect the chip 1275, at least one example of the chip 1275 can include one or more electrical vias 1263a-d electrically routed and coupled to one or more of the grounding pins of the set of pins 1265 at the first, third, and fourth edges 1273, 1269, and 1267, respectively.

Any of the features, components, and/or parts, including the arrangements and configurations thereof shown in FIG. 12 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 12.

FIG. 13 shows a cross-sectional view of a portion of an example of a wearable electronic device 1300 including a display assembly 1306 coupled to a sidewall housing 1302. The display assembly 1306 can include a transparent cover 1312 and a battery 1316 can be disposed within an internal volume defined by the housing 1302. In at least one example, the device 1300 can also include a PCB 1377 disposed between the display assembly 1306 and the battery 1316. An electronic chip 1375, similar to the electronic chips 1175 and 1275 shown in FIGS. 11 and 12, respectively, and described above, can be disposed or coupled to the PCB 1377 between the PCB 1377 and the battery 1316. The device 1300 can also include an electronic component 1343 disposed adjacent the battery 1316.

In at least one example, the chip 1375 can include a first portion 1357 disposed

between the PCB 1377 (and the display assembly 1306) and the electronic component 1343. In at least one example, a temperature sensor 1341 can be disposed on the first portion 1357 and the electronic component 1343 with an open gap 1349, for example an open air gap, between the temperature sensor 1341 and the electronic component 1343. The temperature sensor 1341 can be disposed between the electronic component 1343 and the PCB 1377 (as well as the display assembly 1306). The gap 1349 can serve as a buffer zone between the temperature sensor 1341 and the electronic component 1343 so the two components do not collide during an unintended drop event.

In at least one example, a second portion 1355 of the chip 1375 is disposed between the battery 1316 and the PCB 1377 and therefore between the battery 1316 and the display assembly 1306. In at least one example, the second portion 1355 can include an ambient light sensor 1345. An open gap 1347 can be disposed between the second portion 1355 (and the ambient light sensor 1345 thereof) and the battery 1316 to serve as a buffer zone between the ambient light sensor 1345 and the battery 1316 so the two components do not collide during an unintended drop event.

Any of the features, components, and/or parts, including the arrangements and configurations thereof shown in FIG. 13 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 13.

FIG. 13A shows a cutaway view of a portion of an example of a wearable electronic device 1300 including a sidewall housing 1302 defining an internal volume 1303 and a haptic engine 1305 disposed in the internal volume 1303. In at least one example, the haptic engine 1305 can be part of an alert module or assembly. The haptic engine 1305 can be electrically coupled to one or more other processing components, including processors, memory components, antennas, displays, or other electronic components of the wearable electronic device 1300, and be configured to vibrate, sound, or otherwise produce a haptic and/or audible alert to the user of the device 1300.

In at least one example, the device 1300 includes a system-in-package (SiP) 1307, for example one or more integrated circuits enclosed in a chip carrier or substrate, disposed in the internal volume 1303. In at least one example, the haptic engine 1305 can be a structural bracket securing the SiP 1307 in the internal volume 1303. The haptic engine can include one or more fasteners, for example a screw 1309 passing through the haptic engine 1305 and the SiP 1307 to reduce the total number of fasteners. In this way, the SiP 1307 does not need its own fasteners separate from the haptic engine 1305 operating as the SiP bracket.

In at least one example, the haptic engine 1305, which can also be referred to as a SiP bracket, can include an extension 1311 fastened to the sidewall housing 1302, for example via fastener 1313. The SiP bracket (e.g., the haptic engine 1305) can also include one or more grounding pins, such as grounding pin 1315. Additionally, in at least one example, the haptic engine 1305 can be secured to the SiP 1307 as a bracket via one or more foams disposed there between. In at least one example, the foam can include conductive foam filling a space between the haptic engine 1305 and the SiP 1307.

Any of the features, components, and/or parts, including the arrangements and configurations thereof shown in FIG. 13A can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 13A.

### Pressure Sensor Port

In at least one example of the present disclosure, a wearable electronic device can include a sensor port for measuring external ambient environmental pressures in which the device is disposed and/or operating. The sensor can be disposed internally within the device and a port can extend from the port to the ambient environment through a thickness of a housing of a device. In order to minimize the visual appearance of the port, as well as maximize the volume or size of the external environment with which the port is in communication, the port can be disposed or defined by a portion of the housing between the display assembly and an upper perimeter edge of the housing such that the port extends away from the user. In this way, the user's body does not obscure or cover the port when donning the device. A gap can be formed between the display assembly, for example a transparent cover of the display assembly, and an upper, peripheral edge of the housing. The gap can provide fluid communication between the external environment and a cavity defined between the display assembly and the housing where the port is defined. In this way, the sensor disposed internally can be in communication with the external environment through the port, which is in communication with the external environment through the cavity and the gap.

In at least one example, the cavity can extend substantially all the way around, or completely all the way around, a periphery of the display assembly to maximize the cavity volume in communication with the external environment through the gap. In this way, pressure readings form the sensor can be accurate and repeatable. In addition, the port can be small enough and disposed within the cavity with overhanging housing and display features to visually obscure the port. In this way, the port can be effective for proper pressure measurement taken by the sensor while being visually obscured to maintain the aesthetic appeal of the device from the user's perspective.

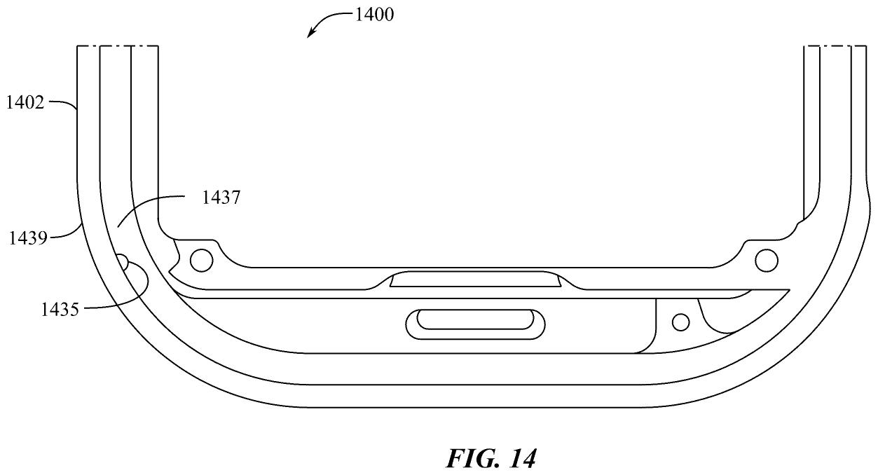

FIG. 14 shows an example of a portion of a housing 1402 of an electronic device 1400. The housing 1402 can define an external surface 1439 and an internal surface 1437. In at least one example, the housing 1402 can define a port 1435. The port 1435 illustrated in FIG. 14 can be an opening to a through-hole extending through a thickness of the housing 1402. In at least one example, the port 1435 can include a circular opening less than about 700 microns in diameters, for example about 500 microns or less in diameter.

In at least one example, the port 1435 can fluidly couple an environmental sensor disposed within the device 1400 to an external environment, for example an ambient environment, of the device 1400. In at least one example, the environmental sensor can include a pressure sensor. In at least one example, the port 1435 can be sized and positioned to be effectively blocked from view to a user viewing the device 1400 externally from the device 1400.

Any of the features, components, and/or parts, including the arrangements and configurations thereof shown in FIG. 14 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 14.

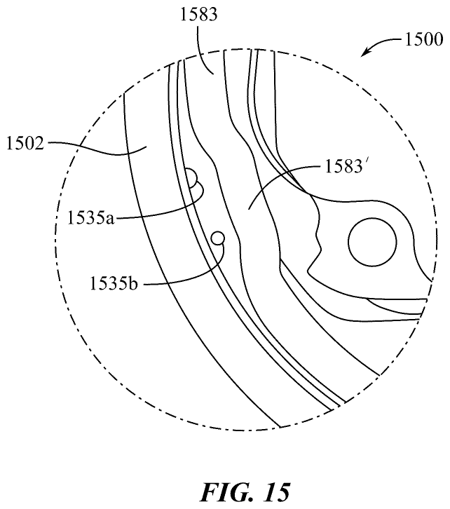

FIG. 15 shows a close-up view of a portion of another example of a device 1500 including a sidewall housing 1502 defining two ports, a first port 1535a and a second port 1535b. The first and/or second ports 1535a-b shown in the example of FIG. 15 can be similar to the port 1435 shown in FIG. 14. Both ports 1535a-b can be defined by the housing 1502 and provide a fluid pathway between an internal volume of the device 1500 and an external environment.

In at least one example, the device 1500 can include a polymer material 1583 (e.g., similar to the polymer material 1083 shown in FIG. 10 and described above) disposed between a display assembly of the device 1500 and the housing 1502. In at least one example, as shown in FIG. 15, the polymer material 1583 can include a jogged section 1583′ to uncover the one or more ports 1535 in order to not occlude the fluid communication with the external environment through the ports 1535a-b.

Any of the features, components, and/or parts, including the arrangements and configurations thereof shown in FIG. 15 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 15.

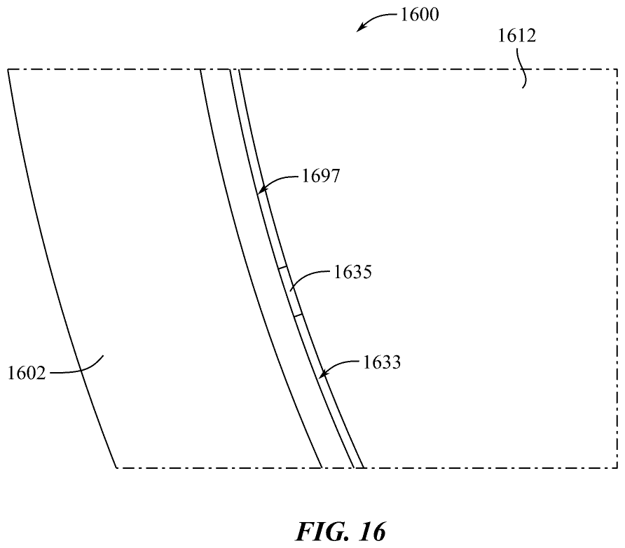

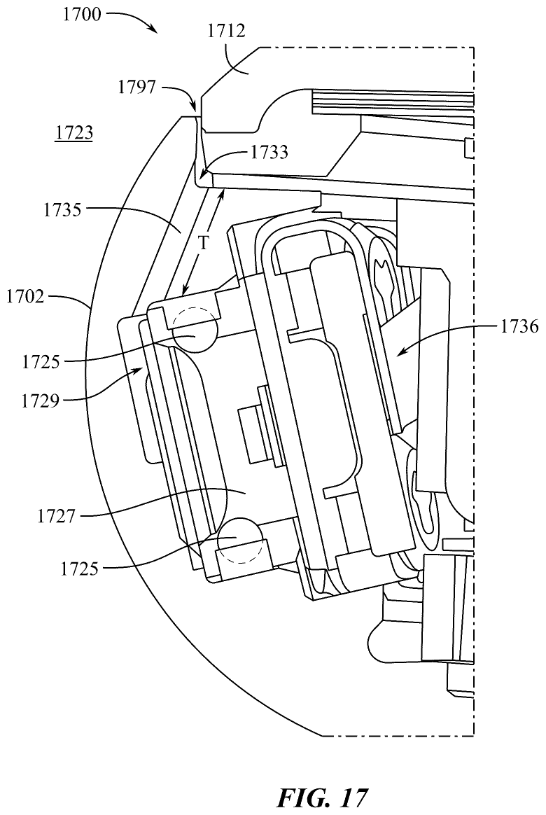

FIG. 16 shows a close-up view of a portion of another example of a device 1600 including a sidewall housing 1602, a transparent cover 1612 of a display assembly, and a port 1635 (e.g., similar to ports 1535a-b and 1435 shown in FIGS. 15 and 14, respectively, and described above). In the illustrated example, the transparent cover 1612 can be spaced apart from the housing 1602 to define a gap 1697, including an open air gap with nothing disposed between the transparent cover 1612 and the housing 1602. In at least one example, the gap 1697 defines a cavity 1633 defined between the housing 1602 and the transparent cover 1612 in fluid communication with an external environment, for example the environment in which the device 1600 is operated, through the gap 1697.

In at least one example, the transparent cover 1612 is part of a front or top cover of the wearable electronic device 1600 and configured to face away from a user's body when the device 1600 is donned. In this way, the port 1635 is facing away from the user's body to avoid being occluded by the user's body during use. In one example, the device 1600 can be configured to be worn on the user's wrist. The front transparent cover 1612 can be opposite a rear cover configured to press against or at least face the user's body when the device 1600 is donned. The port 1635 can be configured to fluidly communicate with the external environment through the gap 1697 between the front, transparent cover 1612 and the housing 1602 as shown such that the user's wrist does not occlude the gap 1697 or the port 1635 when donning the device 1600.

In at least one example, the housing 1602 at least partially overhangs the opening of the port 1635 to obscure a view thereof, as shown in FIG. 16. In one example, the port 1635 is misaligned with the housing 1602, as well as the transparent cover 1612 in some examples, to at least partially obscure a view of the port 1635. In addition, the diameter of the port 1635 being less than about 700 microns, and in some examples as small as about 500 microns or less, reduces the visualization of the port 1635 by the user through the gap 1697. In at least one example, the transparent cover 1612 can also at least partially occlude a view of the port 1635, as illustrated in FIG. 16.

Any of the features, components, and/or parts, including the arrangements and configurations thereof shown in FIG. 16 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 16.

FIG. 17 shows a cross-sectional view of a portion of an example of a wearable electronic device 1700 including a transparent cover 1712 coupled to a sidewall housing 1702 to define a cavity 1733 between the housing 1702 and the transparent cover 1712. The cavity 1733 can be in fluid communication with the external environment 1731 through the gap 1797 between the transparent cover 1712 and the housing 1702. In at least one example, the device 1700 can include a sensor 1727 disposed within an internal volume 1736 of the device 1700. In at least one example, a seal 1725 can be disposed between the sensor 1727 and the housing 1702 to isolate the internal volume 1736 from a sensor volume 1729 defining between the sensor 1727 and the housing 1702. In at least one example, the sensor 1727 can be disposed between the sensor volume 1729 and the internal volume 1736. In at least one example, the sensor 1727 can include a pressure sensor.

In at least one example, the housing 1702 can define a port 1735 extending through a thickness T of the housing 1702 and the sensor volume 1729 can be in fluid communication with the external environment 1723 through the port 1735 and thus also through the cavity (with which the port 1735 is in fluid communication) through the gap 1797 between the transparent cover 1712 and the sidewall housing 1702. In this way, the sensor 1727 can be an environmental sensor configured to detect a condition of the external environment 1723 through the port 1735. For example, the sensor 1727 can be a pressure sensor configured to detect external ambient pressure, or external ambient fluid pressure, for example atmospheric or barometric pressure, of the external environment 1723 through the port 1735.

As noted above, and also illustrated in FIG. 17, the housing 1702 can overhang an opening of the port 1735 defined by the housing 1702 in the cavity 1733 such that the opening of the port 1735 is hidden from view through the gap 1797 from outside the cavity 1733. In at least one example, the port 1735 includes a through-hole or aperture extending from the cavity 1733 to the sensor volume 1729.

Any of the features, components, and/or parts, including the arrangements and configurations thereof shown in FIG. 17 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 17.

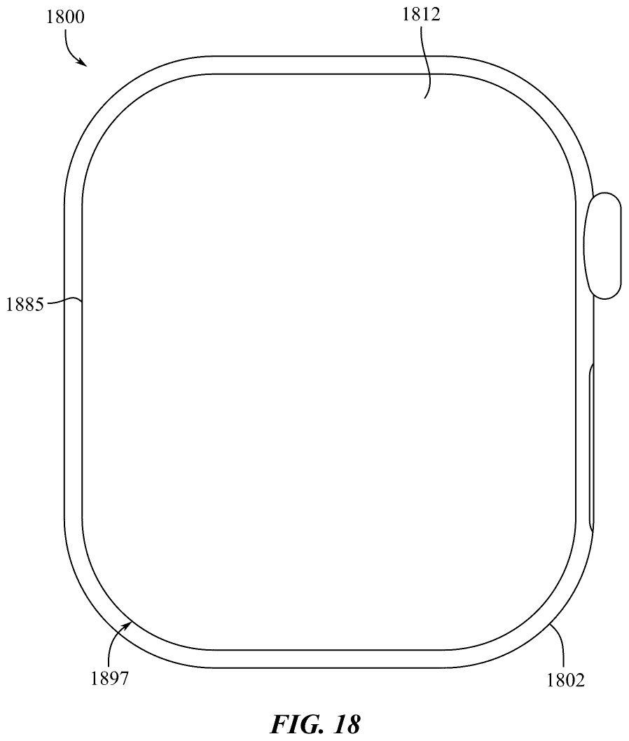

FIG. 18 shows a top, plan view of an example of a wearable electronic device 1800 including a sidewall housing 1802 and a transparent cover 1812. In at least one example, the transparent cover 1812 is separated from the housing 1802 by a gap 1897, for example an open gap similar to other gaps shown in other figures and described above. In at least one example, the gap 1897 can extend around an entire perimeter 1885 of the transparent cover 1812. In such an example, the gap 1897, and the cavity in fluid communication with the external environment through the gap 1897 (e.g., similar to the gap 1797 shown in FIG. 17 and described above), can extend around a perimeter 1885 of the transparent cover 1812 between the transparent cover 1812 and the housing 1802. In this way, the volume of the cavity 1633, 1733 with which the port 1735, 1635, 1535a-b, and 1425 are in fluid communication extends around a perimeter 1885 of the device 1800 to maximize a total volume in fluid communication with the sensor volume 1729, and thus in fluid communication with the external environment 1723, for accurate and repeatable pressure measurements from the pressure sensor 1727.

Any of the features, components, and/or parts, including the arrangements and configurations thereof shown in FIG. 18 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 18.

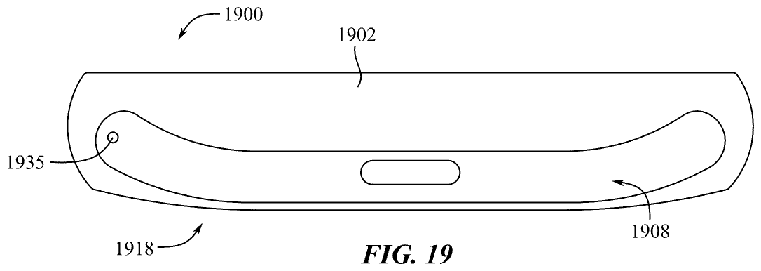

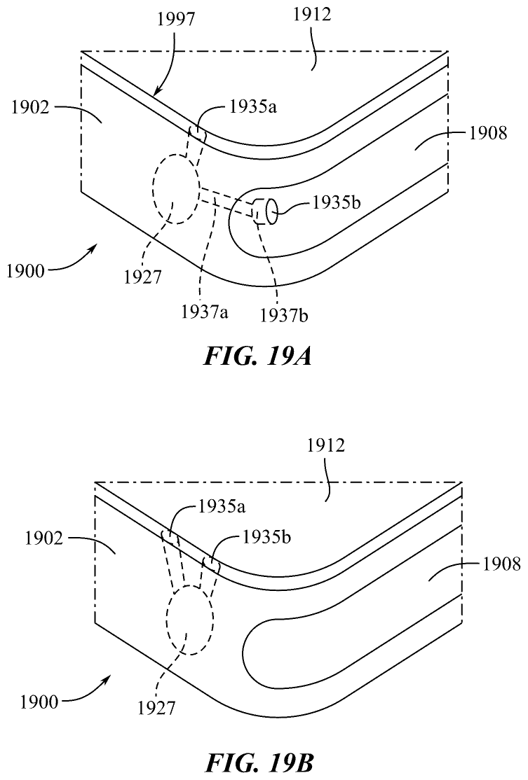

FIG. 19 shows a side view of an example of an electronic device 1900 including a housing 1902 defining a band receiving feature 1908 configured to receive a securement band for securing the electronic device 1900 to a user. In at least one example, the securement band is configured to couple to the housing 1902 via the band receiving feature 1908 and secure a rear cover 1918 of the device 1900 against a body of the user. In at least one example, the device 1900 can include a port 1935 defined by the housing 1902 including an opening defined by the housing within the band receiving feature 1908.

The various ports 1435, 1535a-b, 1635, 1735, and 1935 can be included individually or together in any combination in one or more examples of the electronic devices described herein. In this way, the unobstructed fluid communication between internal sensors of the devices described herein, for example internal pressure sensors, can be maintained with minimal visual aesthetic impact to the devices.

Any of the features, components, and/or parts, including the arrangements and configurations thereof shown in FIG. 19 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 19.

FIG. 19A shows a perspective view of a portion of an example of a wearable electronic device 1900 including a housing 1902 defining a band receiving feature 1908. A transparent cover 1912 of a display assembly of the device 1900 can be positioned to form the gap 1997 between the transparent cover 1912 and the sidewall 1902. In at least one example, a sensor 1927, which can be similar to the sensor 1727 shown in FIG. 17 and described above, can be positioned within the device and can be in communication with an external environment through one or more ports 1935a, 1935b extending from the sensor 1927 to the gap 1997 and the band receiving feature 1908, respectively.

In at least one example, the second port 1935b can include a first portion 1937a having a diameter and/or a cross-sectional shape different from a second portion 1937b. For example, the first portion 1937a can be smaller in diameter than the second portion 1937b. In one example, the second portion 1937b can define an elliptical cross-section (e.g., as opposed to a circular cross-section of the first portion 1937a) such that the opening of the port 1935b at the band receiving feature 1908 is circular even as the port 1935b extends toward the band receiving feature 1908 at an angle. In one example shown in FIG. 19B, the first port 1935a and the second port 1935b can both extend to openings at the gap 1997.

In at least one example, the housing 1902 can be 3D printed to form ports 1935a-b that are curvilinear, snaking, or otherwise irregularly formed, for example ports having corners, turns, or other non-linear features. The ports 1935a-b shown in FIGS. 19-19B, as well as ports shown in other figures and described herein, can be formed and included within the device 1900 in any combination such that one or multiple ports can be utilized to expose the sensor 1927 to the external environment of the device 1900. In one example, one or more ports 1935a-b can extend to a speaker port of the device 1900. In one example, one or more ports 1935a-b can extend to a rear cover of the device 1900.

Any of the features, components, and/or parts, including the arrangements and configurations thereof shown in FIGS. 19A and 19B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 19A and 19B.

### Speaker Assembly

In at least one example of the present disclosure, a wearable electronic device can include a speaker module disposed within the device and at least partially within a cavity formed into a thickness of a housing of the device. In at least one example, a diaphragm of the speaker module can be integrally formed with a radial seal disposed between the housing (within the cavity) and a frame of the speaker module. The frame can be formed of metal to provide strong, stiff, structural support to the diaphragm and speaker driver. The seal can be disposed between a surface of the cavity parallel with the diaphragm and/or the driver such that when a yoke coupling the driver and the frame to the housing presses the seal between the frame and the housing, the seal does not press radially inward on the frame.

Instead, the seal is pressed between the frame and housing to transfer a force from the yoke into the housing with a directional force orthogonal to a plane in which the radial seal sits. In this way, the yoke and the seal do not press inward on the frame to compromise the shape and proper functioning of the diaphragm. Rather, the movement of the diaphragm is along an axis parallel to the force from the yoke, through the frame, and against the housing within the cavity. In this way, the metal frame can be thinner while maintaining stiffness for adequate structural support due to the metal material and the seal can properly seal off the internal volume of the device to prevent leaks and debris from passing into and around the speaker module.

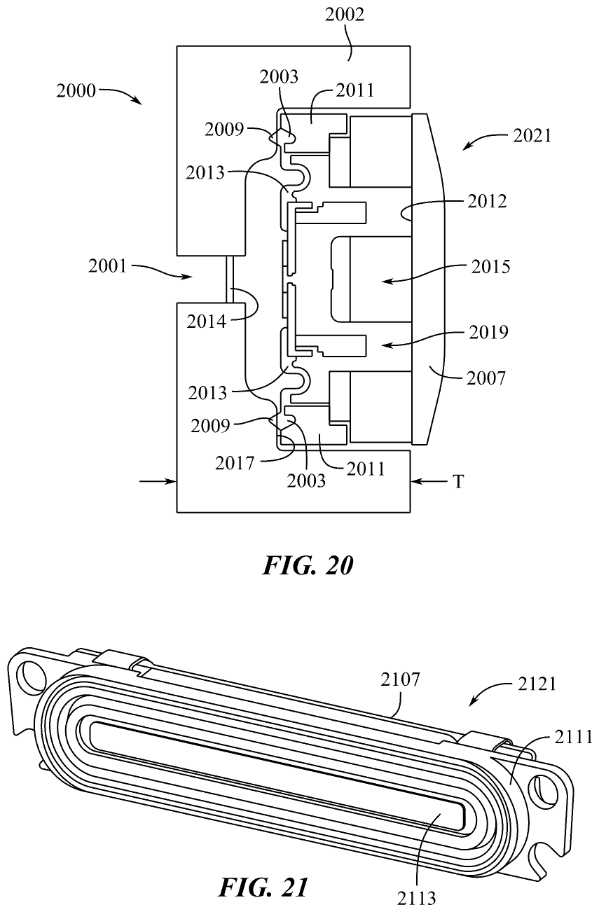

FIG. 20 shows a cross-section view of a portion of an example of an electronic device including a housing 2002 can a speaker assembly or module 2021 coupled to the housing 2002. In at least one example, the housing 2002 can be a sidewall housing defining a recess 2019 extending into a thickness T of the housing 2002 including a recessed surface 2017 defining the recess 2019. The recessed surface 2017 can be within the thickness T of the housing 2002. In at least one example, the device 2000 can include a speaker module 2015, including a speaker assembly, disposed at least partially within the recess 2019.

In at least one example, the speaker module 2015 can include an acoustic diaphragm 2013, a frame 2011 surrounding the acoustic diaphragm 2013, a seal 2009 extending between the diaphragm 2013 and the frame 2011, and a yoke 2007 coupled to the housing 2002 and configured to press the seal 2009 between the frame 2011 and the recessed surface 2017. In at least one example, the recessed surface 2017 can be a sealing surface against which the seal 2009 is pressed between the sealing surface and the frame 2011. In at least one example, the seal 2009 is configured to prevent sound, fluid, and/or air from crossing the seal 2009 between the recessed surface 2017 and the frame 2011.

In at least one example, the frame 2011 is coupled to the speaker driver 2005. In at least one example, the frame 2011 can include metal and a compression force can be configured to transfer through the frame 2011 from the yoke 2007 to the seal 2009. The parallel or generally parallel nature of the recessed surface 2017 relative to an internal surface 2012 of the yoke 2007, as well as the seal 2009 disposed between the recessed surface 2017 and the frame 2011, prevents any forces being directed radially inward toward the center of the diaphragm 2013, thereby putting less or no compression force inward on the diaphragm 2013 or the frame 2011, and thus on the driver 2005 or any other components. In this way, speaker quality and longevity can be sustained and improved.

In at least one example, the diaphragm 2013 can include a mechanical interlock feature 2003 disposed between the frame 2011 and the recessed surface 2017. In at least one example, the seal 2009 can include the mechanical interlock feature 2003 of the diaphragm, including complimentary shapes of the seal 2009 and the frame 2011, which couples the seal 2009 to the frame 2011. In such an example, the seal 2009 can be integrally formed with the diaphragm 2013 as a single, unitary piece including the mechanical interlock feature 2003. In at least one example, the mechanical interlock feature 2003 can be compressed between the recessed surface 2017 and the frame 2011.

In at least one example, the speaker driver 2015 can include an electromagnetic driver coupled to an internal surface 2012 of the yoke 2007. As noted above, the internal surface 2012 of the yoke 2007 can be parallel of generally parallel to the recessed surface 2017.

In at least one example, seal 2009 can include a first material and the frame 2011 can include a second material harder and/or stiffer than the first material. For example, as noted above, the frame 2011 can include a metal material and the seal 2009 can include silicone or another polymer material softer than the metal of the frame 2011.

In at least one example, the housing 2002 defines an aperture 2001 in fluid communication with the recess 2019. In at least one example, the recess 2019 can be a cavity and the aperture 2001 can be in fluid communication with the cavity. In at least one example, the device 2000 can include a speaker mesh 2014 disposed over or across the aperture 2001. In such an example, the speaker mesh 2014 can be coupled to the housing 2002.

Any of the features, components, and/or parts, including the arrangements and configurations thereof shown in FIG. 20 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 20.

FIG. 21 shows a perspective view of an example of a speaker module 2121 similar to the speaker module 2021 shown in FIG. 20 and described above. The speaker module 2121 shown in FIG. 21 can include a diaphragm 2113 coupled to a frame 2111 and a yoke 2107 coupled to the frame 2111. The frame 2111 can be disposed between the diaphragm 2113 and the yoke 2107.

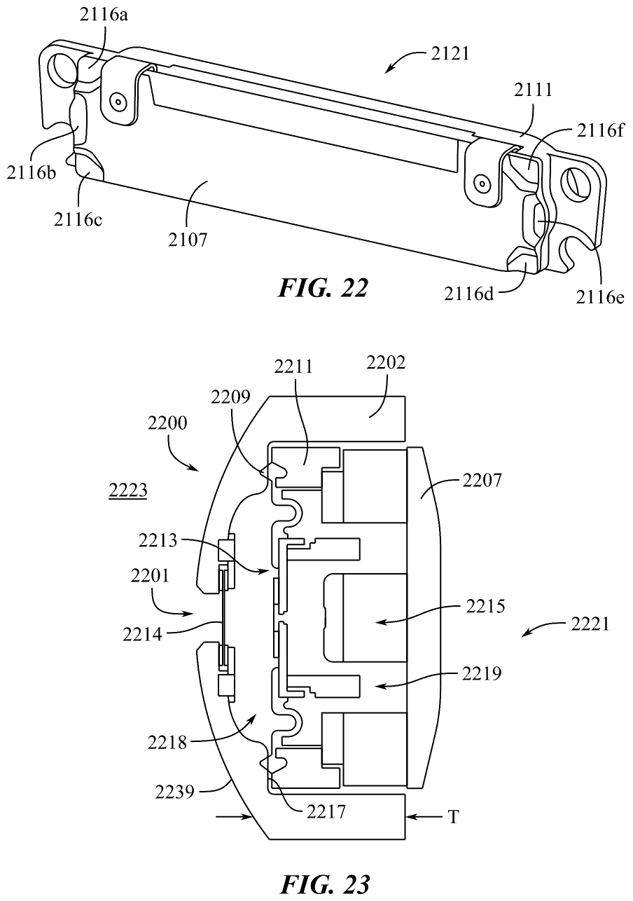

FIG. 22 shows another perspective view of the speaker module 2121 shown in FIG. 21, including the yoke 2107 coupled to the frame 2111. In at least one example, the yoke 2107 can be welded to the housing and/or frame 2111 around a perimeter of the recess 2019 and/or the frame 2111 at one or more weld spots 2116a-f.

Any of the features, components, and/or parts, including the arrangements and configurations thereof shown in FIG. 22 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 22.

FIG. 23 shows another example of a speaker module 2221 including a housing 2202 can a speaker assembly or module 2221 coupled to the housing 2202. In at least one example, the housing 2202 can be a sidewall housing defining a recess 2219 extending into a thickness T of the housing 2202 including a recessed surface 2217 defining the recess 2219. The recessed surface 2217 can be within the thickness T of the housing 2202. In at least one example, the device 2200 can include a speaker module 2215, including a speaker assembly, disposed at least partially within the recess 2219.

In at least one example, the speaker module 2215 can include an acoustic diaphragm 2213, a frame 2211 surrounding the acoustic diaphragm 2213, a seal 2209 extending between the diaphragm 2213 and the frame 2211, and a yoke 2207 coupled to the housing 2202 and configured to press the seal 2209 between the frame 2211 and the recessed surface 2217. In at least one example, the recessed surface 2217 can be a sealing surface against which the seal 2209 is pressed between the sealing surface and the frame 2211. In at least one example, the seal 2209 is configured to prevent sound, fluid, and/or air from crossing the seal 2209 between the recessed surface 2217 and the frame 2211.

In at least one example, the housing 2202 defines an aperture 2201 in fluid communication with the recess 2219. In at least one example, the recess 2219 can be a cavity and the aperture 2201 can be in fluid communication with the cavity. In at least one example, the device 2200 can include a speaker mesh 2214 disposed over or across the aperture 2201. In such an example, the speaker mesh 2014 can be coupled to the housing 2202 and serve as a barrier between the external environment 2223 of the device 2200 and an internal speaker volume 2218 between the diaphragm 2213 and the mesh 2214. In at least one example, the housing 2202 can define a curved external surface 2239 of the device 2200.

Any of the features, components, and/or parts, including the arrangements and configurations thereof shown in FIG. 23 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 23.

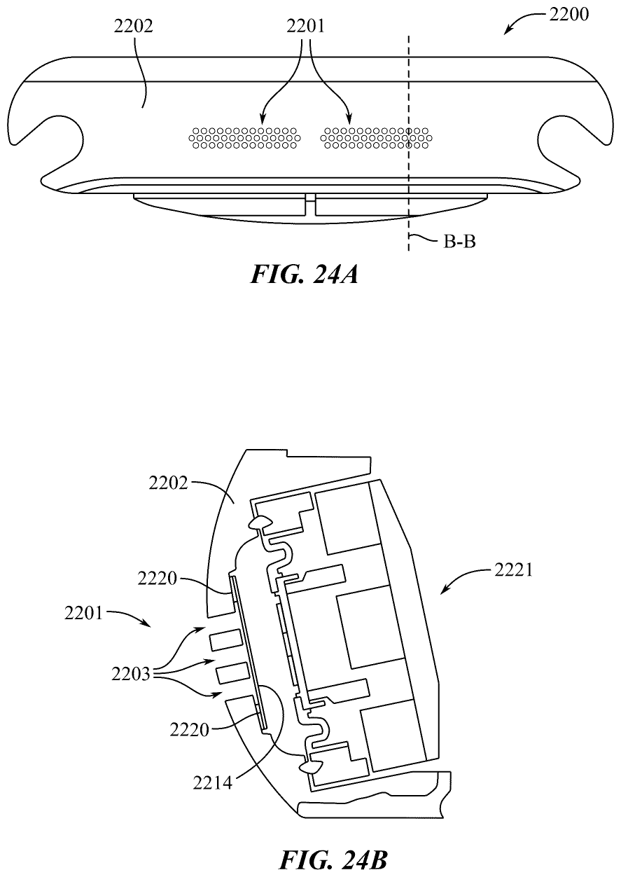

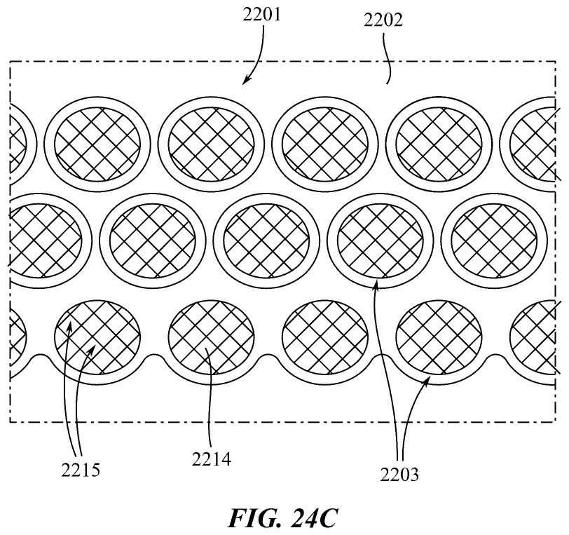

FIG. 24A shows a side view of an example of the device 2200 including a housing 2202 defining one or more speaker apertures or speaker ports 2201. In at least one example, the speaker ports 2201 can be disposed on a side of the device 2200 corresponding to a speaker or multiple speakers disposed therein. The speaker ports 2201 can be formed of perforations including a plurality of holes formed through a thickness of the housing 2202. In at least one example, the perforations can be machined, etches, punched, molded, or otherwise formed in the housing 2202.

FIG. 24B shows a cross-sectional view of the device 2200 along the plane B-B indicated in FIG. 24A. As shown in the cross-sectional view, the speaker module 2221 can be similar to the speaker module 2221 shown in FIG. 24. In the example shown in FIG. 24A, the speaker port 2201 can include perforations 2203 formed through a thickness of the housing 2202. In at least one example, the speaker mesh 2214 can be disposed against, or directly against, the housing 2202 on an internal surface thereof as shown. In one example, the mesh 2214 can be adhered to the housing 2202 via adhesives 2220, for example heat activate films, pressure sensitive adhesives, glue, epoxy, or other securement mechanisms. In such an example, the speaker module 2221 can be positioned closer to the inner surface of the housing 2202 to reduce space taken up inside the device 2200 with the mesh 2214 disposed against the internal surface of the housing 2202.

FIG. 24C shows a close-up view of the speaker port 2201 shown in the side view of FIG. 24A. As shown, the speaker port 2201 can include a plurality of perforations 2203 formed through the housing 2202. The mesh 2214 is seen through each perforation 2203 against the inner surface of the housing 2202. The mesh 2214 can include a woven wire structure forming mesh pores 2215 between adjacent wires. The sized and locations of the perforations 2203 and mesh pores 2215 can be such that water ejection and speaker protection are optimized. In at least one example, the perforations 2203 of the speaker port 2201 include diameters between about 100 microns and about 700 microns. In one example, the diameter of the perforations 2203 can be between about 200 microns and about 600 microns, or between about 300 microns and about 500 microns, for example about 400 microns.

In at least one example, the mesh pores 2215 can be defined by open dimensions (e.g., dimensions across each pore 2215) of between about 40 microns and about 70 microns. In one example, the open dimensions of the pores 2215 can be between about 50 microns and about 65 microns, for example about 53 microns or about 63 microns. The relative dimensions and number of the larger perforations 2203 and the smaller mesh pores 2215 disposed against the perforations 2203 can encourage water and debris ejection from the internal volume of the device 220 through the speaker port 2201 while not affecting the sound quality of the speaker module 2221 as heard through the speaker port 2201.

Any of the features, components, and/or parts, including the arrangements and configurations thereof shown in FIGS. 24A-24C can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 24A-24C.

### Band Release Mechanism

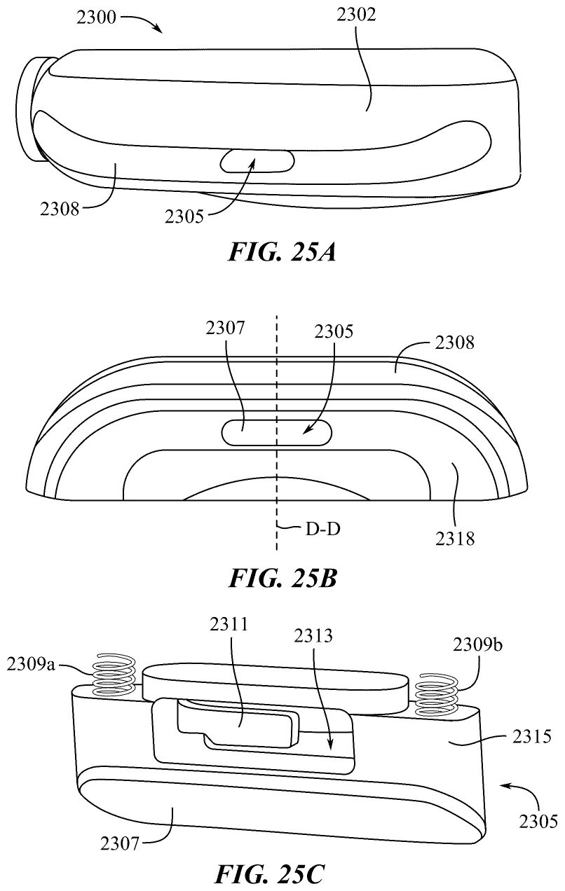

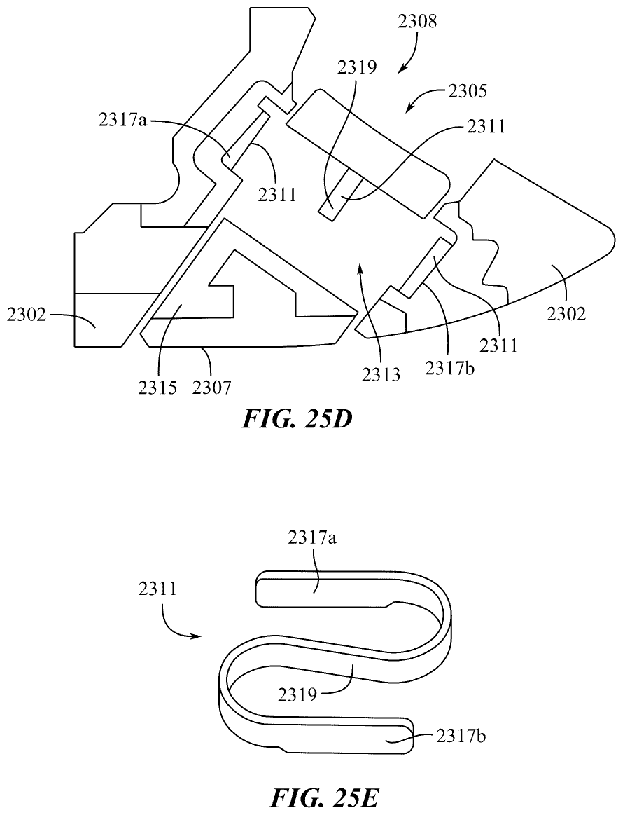

FIG. 25A shows a sideview of an example of a wearable electronic device 2400 including a housing 2302 defining a band receiving feature 2308 similar to other band receiving features described herein. The band receiving feature 2308 can be formed to receive a securement band configured to secure the device 2300 to a user's body. In at least one example, the band receiving feature 2308 can include a band release mechanism 2305 disposed within and/or at least partially defining the band receiving feature 2308. The release mechanism 2305 can be movable to expel the band when secured to the housing 2302 at the band receiving feature 2308. For example, the release mechanism 2305 can be movable between a flush, sub-flush, and/or protruding configuration relative to a surface of the housing 2302 defining the band receiving feature 2308. The movement or different positions of the release mechanism 2305 can lock and release the band from the band receiving feature 2308 during use.

In at least one example, the release mechanism 2305 can extend through the device 2300 such that a manipulation surface 2307 of the release mechanism 2305 shown in FIG. 25B can be accessible on a lower portion of the device 2300, such as for example as part of the rear cover 2318 of the device 2300. The user can press the release mechanism 2305 via the manipulation surface 2307 thereof to move release and/or lock the securement band from the band receiving feature 2308.

FIG. 25C shows a perspective view of an example of the release mechanism 2305 isolated from the rest of the device 2300. The release mechanism 2307 can include a first portion defining the manipulation surface 2307 and formed of a first material, for example a conductive material such as a metal. The release mechanism 2307 can also include a second portion 2315 coupled with or molded to the first portion. The second portion 2315 can include a non-conductive material so as not to interfere with electrical signals, antennas, and the like of the device 2300 as the release mechanism 2305 is manipulated and moved relative to conductive portions of the housing 2302.

In at least one example, the second, non-conductive portion 2315 can define an aperture 2313 in which a retention clip 2311 is disposed. The retention clip 2311 can secure the release mechanism 2305 within the housing 2302 while allowing the desired movement. The release mechanism 2305 can also include one or more biasing members, such as coil springs 2309a and 2309b to bias the release mechanism 2305 into a resting position when the securement band is locked in the band receiving feature 2308. The user can manipulate the release mechanism 2305 via the manipulation surface 2307 to overcome the biasing force of the biasing members.

FIG. 25D shows a cross-sectional view of the device 2300 along the plane D-D indicated in FIG. 25B. The cross-sectional view shows the release mechanism 2305 within the housing 2302, with the manipulation surface 2307 and second portion 2315 defining the aperture 2313 in which the retention clip 2311 is disposed. The housing 2302 defines the band receiving feature 2308 in which a securement band of the device 2300 can be locked and released. The retention clip 2311 can extend across the aperture 2313 to engage recessed features of the housing 2302 to keep the release mechanism 2305 in position while moving and/or while static. In at least one example, the opposing ends 2317a and 2317b of the retention clip 2311 can be formed of, and/or include, non-conductive materials so as not to interfere with electrical signals of the housing 2302, antennas, or other electrical signals of the device 2300. A middle portion 2319 of the retention clip 2319 can engage the release mechanism 2305 while the ends 2317a-b engage the housing 2302 as shown.

FIG. 25E shows an isolated, perspective view of an example of the retention clip 2311. The clip 2311 can include opposing ends 2317a-b and a middle portion 2319, as noted above. The ends 2317a-b can include non-conductive materials and the middle portion 2319 can include conductive or non-conductive materials. In at least one example, the retention clip 2305 is serpentine in shape to accomplish its retaining functionality. The serpentine shape allows for a single piece to include opposing ends 2317a-b that engage the housing, and a middle portion 2319 that engages the release mechanism 2305 as the release mechanism 2305 is manipulated and moved relative to the housing 2302.

Any of the features, components, and/or parts, including the arrangements and configurations thereof shown in FIGS. 25A-25E can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 25A-25E.

### Dial

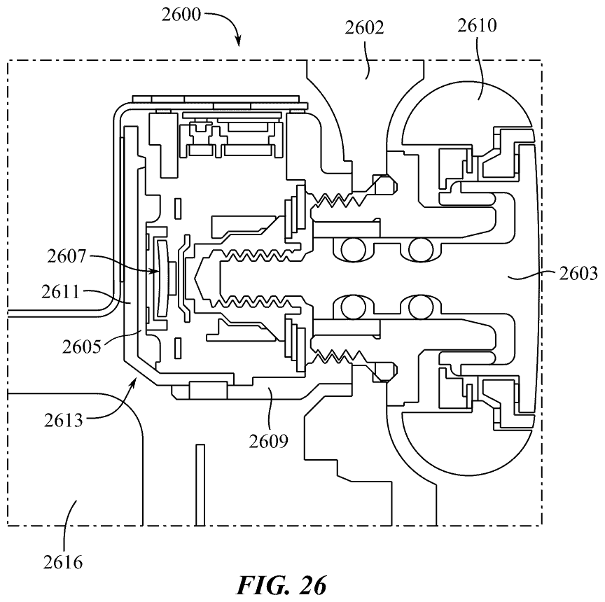

FIG. 26 shows a cross-sectional view of a portion of an example of a wearable electronic device 2600, including a depressible dial button 2610 extending through the housing 2602. The button 2610 can include a rotatable dial 2603 and an electrical switch 2607 inside the device 2600. In one example, a bucket 2609 and a shear plate 2605 of the button 2610 assembly are reduced in size and are positioned to make space for a larger battery 2616. In addition, a button bracket 2611 can form a beveled corner feature 2613 to provide more space for the battery 2616.

Any of the features, components, and/or parts, including the arrangements and configurations thereof shown in FIG. 26 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 26.

To the extent applicable to the present technology, gathering and use of data available from various sources can be used to improve the delivery to users of invitational content or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, TWITTER® ID's, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide mood-associated data for targeted content delivery services. In yet another example, users can select to limit the length of time mood-associated data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not intended to be exhaustive or to limit the embodiments to the precise forms disclosed. It will be apparent to one of ordinary skill in the art that many modifications and variations are possible in view of the above teachings.

What is claimed is:

1. An electronic device, comprising:
a housing;
a display assembly coupled to the housing;
a cover coupled to the housing opposite the display assembly, the housing disposed between the cover and the display assembly, the cover comprising:
a non-metallic portion including a perimeter edge, the non-metallic portion defining an external surface; and
a metal portion surrounding the non-metallic portion between the non-metallic portion and the housing;
an electrical conductor extending from the external surface defined by the non-metallic portion and around the perimeter edge between the non-metallic portion and the metal portion; and
an insulator disposed between the metal portion and the electrical conductor.

2. The electronic device of claim 1, wherein;
the housing defines an internal volume;
the electronic device further comprises:
an electronic component disposed in the internal volume; and
an electrode disposed on the external surface defined by the non-metallic portion; and
the electrical conductor electrically couples the electrode to the electronic component.

3. The electronic device of claim 1, wherein the insulator comprises a plastic insulator disposed between the metal portion and the perimeter edge.

4. The electronic device of claim 1, wherein the insulator comprises an insulative physical vapor deposition (PVD) coating extending around the perimeter edge between the electrical conductor and the metal portion.

5. The electronic device of claim 4, wherein the PVD coating comprises at least one of silicon nitride, silicon oxynitride, or silicon dioxide.

6. The electronic device of claim 1, wherein the insulator defines the external surface.

7. The electronic device of claim 1, wherein the non-metallic portion comprises a transparent window.

8. The electronic device of claim 7, the transparent window comprises sapphire.

9. The electronic device of claim of 7, further comprising a light emitter disposed in an internal volume defined by the housing and configured to transmit light through the transparent window.

10. An electronic display device, comprising:
a housing;
a display assembly coupled to the housing;
a cover coupled to the housing opposite the display assembly, the cover comprising:
a transparent window including a perimeter edge, the transparent window defining an external surface; and
a metal portion surrounding the transparent window;
an electrical conductor extending from the external surface defined by the transparent window and around the perimeter edge between the transparent window and the metal portion; and
a plastic insulator surrounding the transparent window between the metallic portion and the electrical conductor.

11. The electronic display device of claim 10, wherein the plastic insulator extends around a perimeter of the transparent window.

12. The electronic display device of claim 11, wherein the plastic insulator defines the external surface.

13. The electronic display device of claim 10, further comprising:
a sensor assembly disposed within the housing; and
an electrode disposed on the external surface defined by the transparent window, the electrode electrically coupled to the sensor assembly via the electrical conductor.

14. The electronic display device of claim 10, wherein the transparent window comprises sapphire.

15. The electronic display device of claim 10, wherein:
the housing comprises metal;
the plastic insulator is a first plastic insulator; and
the electronic display device comprises a second plastic insulator disposed between the metal portion and the housing.

16. The electronic display device of claim 15, further comprising an antenna assembly including the metal portion.

17. A display device, comprising:
a housing;
a display assembly coupled to the housing;
a cover coupled to the housing opposite the display assembly, the cover comprising:
a non-metallic portion including a perimeter edge, the non-metallic portion defining an external surface; and
a metal portion disposed between the non-metallic portion and the housing;
an electrical conductor extending from the external surface defined by the non-metallic portion and around the perimeter edge between the non-metallic portion and the metal portion; and
an electrically insulating coating extending on the perimeter edge between the electrical conductor and the metal portion.

18. The display device of claim 17, wherein the electrically insulating coating comprises a ceramic material.

19. The display device of claim 18, further comprising an electrode disposed on the external surface of the non-metallic portion, the electrode in electrical communication with an electronic component within the display device via the electrical conductor.

20. The display device of claim 17, wherein:
the non-metallic portion comprises a transparent window; and
the display device further comprises a light emitter configured to transmit light through the transparent window.
